SEQUENCE LISTING (41 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 4 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 6 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 7 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 8 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 9 - DNA, 540 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 10 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 11 - DNA, 54 bp, Homo sapiens
  CDS (1)..(54)
atggctcccttagccgaagtcgggggctttctgggcggcctggagggcttgggc

SEQ ID NO: 12 - protein, 18 aa, Homo sapiens
MAPLAEVGGFLGGLEGLG

SEQ ID NO: 13 - DNA, 63 bp, Homo sapiens
  CDS (1)..(63)
gagcggccgccgctgctgggcgagcgcaggagcgcggcggagcggagcgcgcgcggcgggccg

SEQ ID NO: 14 - protein, 21 aa, Homo sapiens
ERPPLLGERRSAAERSARGGP

SEQ ID NO: 15 - DNA, 63 bp, Homo sapiens
  CDS (1)..(63)
cgcaggtattttgtggcacttaacaaagacggaactccaagagatggcgccaggtccaagagg

SEQ ID NO: 16 - protein, 21 aa, Homo sapiens
RRYFVALNKDGTPRDGARSKR

SEQ ID NO: 17 - DNA, 60 bp, Homo sapiens
  CDS (1)..(60)
cctagaccagtggatccagaaagagttccagaattgtacaaggacctactgatgtacact

SEQ ID NO: 18 - protein, 20 aa, Homo sapiens
PRPVDPERVPELYKDLLMYT

SEQ ID NO: 19 - DNA, 51 bp, Homo sapiens
  CDS (1)..(51)
atgaacgacaagggcgagctgtacggcagcgagaagctgaccagcgagtgc

SEQ ID NO: 20 - protein, 17 aa, Homo sapiens
MNDKGELYGSEKLTSEC

SEQ ID NO: 21 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 22 - protein, 211 aa, Homo sapiens [sequence omitted]

SEQ ID NO: 23 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 24 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 26 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 27 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 28 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 29 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 30 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 31 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 32 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 33 - DNA, 402 bp, Homo sapiens
  CDS (1)..(402)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgac

SEQ ID NO: 34 - protein, 134 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVD

SEQ ID NO: 35 - DNA, 447 bp, Homo sapiens
  CDS (1)..(447)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 36 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 37 - DNA, 396 bp, Homo sapiens
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagacca

SEQ ID NO: 38 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRP

SEQ ID NO: 39 - DNA, 396 bp, Homo sapiens
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagacca

SEQ ID NO: 40 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRP

SEQ ID NO: 41 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

(19) United States
(12) Patent Application Publication
(10) Pub. No.: US 2008/0287349 A1
(43) Pub. Date: Nov. 20, 2008

(54) Fibroblast growth factor 20 and methods of use thereof

(76) Inventors: Jeffrey Peterson, Guilford, CT (US); Paul Sciore, Calgary (CA); Erik Gunther, Branford, CT (US); Marie Ruiz-Martinez, Bethany, CT (US); Galina Chernaya, Madison, CT (US); William J. LaRochelle, Madison, CT (US); Michael E. Jeffers, Branford, CT (US); Henri Lichenstein, Guilford, CT (US); Peter Mezes, Old Lyme, CT (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 10/980,458

(22) Filed: Nov. 3, 2004

Related U.S. Application Data
(63) Continuation-in-part of application No. 10/842,206, filed on May 10, 2004, now abandoned.
(60) Provisional application No. 60/469,353, filed on May 9, 2003.

(51) Int. Cl. A61K 38/17; A61K 31/711; C07H 21/04; C12N 15/63; C12N 5/06; C12N 1/00; C12P 21/00; C07K 14/00; A61P 19/00; A61P 19/02

(52) U.S. Cl. 514/12; 514/44; 536/23.5; 435/320.1; 435/243; 435/325; 435/69.1; 530/350

(57) ABSTRACT

The present invention relates to compositions and methods for preventing and treating a disease (e.g., a joint disease, ischemic stroke, hemorrhagic stroke, trauma, spinal cord damage, heavy metal or toxin poisoning, or neurodegenerative diseases). More particularly, the present invention provides methods for preventing and/or treating a disease (e.g., a joint disease, ischemic stroke, hemorrhagic stroke, trauma, spinal cord damage, heavy metal or toxin poisoning, or neurodegenerative diseases) by using compositions comprising FGF-20, a fragment, a derivative, a variant, a homolog, or an analog thereof.

This application is a continuation-in-part of the U.S. patent application Ser. No. 10/842,206, filed May 10, 2004, which claims the benefit of priority of U.S. Provisional Application Ser. No. 60/469,353, filed May 9, 2003. The content of each application is incorporated herein by reference in its entirety.

### 1. FIELD OF THE INVENTION

The present invention relates to compositions and methods of prevention and/or treatment of certain disorders (e.g., stroke, neurodegenerative diseases, trauma, and joint diseases (e.g., osteoarthritis and rheumatoid arthritis)). More particularly, the present invention relates to compositions comprising FGF-20, a fragment, a derivative, a variant, a homolog, or an analog thereof, and their uses in preventing and treating a disorder, such as but is not limited to, stroke, a neurodegenerative disease, and a joint disease (e.g., osteoarthritis and rheumatoid arthritis), as well as their uses in wound healing.

### 2. BACKGROUND OF THE INVENTION

2.1 Arthritis: Osteoarthritis and Rheumatoid Arthritis

Osteoarthritis (“OA”) is a degenerative joint disease and a frequent cause of joint pain that affects a large and growing population. OA is estimated to be the most common cause of disability in adults. The disease typically manifests itself in the 2nd to 3rd decades, with most people over forty years exhibiting some pathologic change in weight bearing joints, although the change may be asymptomatic. A systematic review of incidence and prevalence of OA of the knee in people older than 55 years in the United Kingdom reported an incidence of 25 percent per year, a prevalence of disability of 10 percent, and severe disability in about two to three percent. The National Health and Nutrition Examination Survey (Center for Health Statistics, Centers for Disease Control and Prevention) found the prevalence of this disease to be over 80 percent in people over age 55, compared to less than 0.1 percent in those aged 25 to 34 years old. OA results from a complex interplay of multiple factors, including joint integrity, genetics, local inflammation, mechanical forces, and cellular and biochemical processes. Characteristic features of the disease are degradation of articular cartilage, hypertrophy of bone at the margins, and changes in the synovial membrane, typically accompanied by pain and stiffness of the joint. For the majority of patients, OA is linked to one or more factors, such as aging, occupation, trauma, and repetitive and small insults over time. The pathophysiologic process of OA is almost always progressive.

There are several well-established treatment modes for OA, ranging from non-pharmaceutical to pharmaceutical intervention. Non-pharmaceutical interventions include behaviour modification, weight loss, exercise, walking aids, avoidance of aggravating activities, as well as joint irrigation, and arthroscopic and surgical interventions. Current pharmaceutical interventions include nonsteroidal antiinflammatory drugs, intraarticular corticosteroids, and colchicine. In addition, FGF-18 has been shown to repair damaged cartilage in a rat meniscal tear model for OA (See Paper #0199, 50th Annual Meeting of the Orthopaedic Research Society, San Francisco Calif., 2004).

However, satisfactory treatment of OA is an unmet medical need, as existing therapeutics have not been successful in curtailing the incidence or the severity of the disease. Consequently, a therapeutic that can successfully treat osteoarthritis has the beneficial effects of decreasing morbidity, while potentially saving the healthcare system millions of dollars in costs associated with invasive surgical procedures, disability and ancillary support services.

2.2 Fibroblast Growth Factors

The fibroblast growth factor (“FGF”) family has more than 20 members, each containing a conserved amino acid core (see, e.g., Powers et al., Endocr. Relat. Cancer, 7(3):65-197 (2000)). FGFs regulate diverse cellular functions such as growth, survival, apoptosis, motility, and differentiation (see, e.g., Szebenyi et al., Int. Rev. Cytol., 185:45-106 (1999)). Members of the FGF family are involved in various physiological and pathological processes during embryogenesis and adult life, including morphogenesis, limb development, tissue repair, inflammation, angiogenesis, and tumor growth and invasion (see, e.g., Powers et al., Endocr. Relat. Cancer, 7(3):165-197 (2000); and Szebenyi et al., Int. Rev. Cytol. 185:45-106 (1999)).

FGFs transduce signals via high affinity interactions with cell surface tyrosine kinase FGF receptors (FGFRs). These FGF receptors are expressed on most types of cells in tissue culture. For example, FGF receptor-1 (FGFR-1), which shows the broadest expression pattern of the four known FGF receptors, contains at least seven tyrosine phosphorylation sites. A number of signal transduction molecules are affected by binding with different affinities to these phosphorylation sites.

FGFs also bind, albeit with low affinity, to heparin sulfate proteoglycans (HSPGs) present on most cell surfaces and extracellular matrices (ECM). Interactions between FGFs and HSPGs may serve to stabilize FGF/FGFR interactions, and to sequester FGFs and protect them from degradation (Szebenyi and Fallon, Int. Rev. Cytol. 185:45-106. (1999)).

Glia-activating factor (“GAF”), another FGF family member, is a heparin-binding growth factor that was purified from the culture supernatant of a human glioma cell line. See, Miyamoto et al., Mol. Cell Biol. 13(7): 4251-4259 (1993). GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N-terminus like those in acidic FGF and basic FGF.

Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N-terminus with respect to those predicted by the cDNA sequence, except the initiation methionine. The rat FGF-9 cDNA was also cloned, and the structural analysis indicated that the FGF-9 gene is highly conserved.

Through a homology-based genomic mining process, a novel human FGF, FGF-20, was discovered. See U.S. patent application Ser. No. 09/494,585, filed Jan. 13, 2000, and Ser. No. 09/609,543, filed Jul. 3, 2000, the disclosure of each references is incorporated herein by reference. The amino acid sequence of FGF-20 shows close homology with human FGF-9 (70% identity) and FGF-16 (64% identity).

FGF-20 and its variants belong to the FGF family that regulates proliferation (see U.S. application Ser. No. 10/174,394, which is incorporated herein by its entirety). The identification of a polymorphism (CG53135-12) in the gene encoding FGF-20 in humans, and a method for identifying individuals who are carriers of the genetic risk-altering factor for OA have been described in U.S. application Ser. No. 10/702,126 (“the '126 application”), which is incorporated herein by its entirety. The '126 application describes a DNA-based diagnostic test for identifying individuals with increased risk for OA and resultant musculoskeletal complications.

Citation or discussion of a reference herein shall not be construed as an admission that such is prior art to the present invention.

### 3. SUMMARY OF THE INVENTION

The present invention provides methods of preventing or treating a disease (e.g., stroke, joint diseases, neurodegenerative diseases, and trauma) comprising administering to a subject in need thereof a composition comprising one or more CG53135 proteins.

In accordance with the present invention, the diseases to be prevented or treated include, but are not limited to, joint diseases (non-limiting examples being arthritis, osteoarthritis, joint pathology, ligament and tendon injuries, and meniscal injuries), ischemic stroke, hemorrhagic stroke, trauma, spinal cord damage, heavy metal or toxin poisoning, and neurodegenerative diseases (non-limiting examples being Alzheimer's, Parkinson's Disease, Amyotrophic Lateral Sclerosis, Huntington's Disease).

The invention provides a prophylactic treatment with CG53135 wherein an injury that predisposes the subject to osteoarthritis has occurred but the cartilage is intact.

The invention also provides a therapeutic treatment with CG53135 wherein intrinsic or extrinsic factors (e.g. genetic predisposition or meniscal injury, respectively) have led to osteoarthritic changes and cartilage damage.

Pharmaceutical formulations and kits are also provided by the instant invention.

3.1 Terminology

As used herein, the term “CG53135”, refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs. In a preferred embodiment, a CG53135 protein retains at least some biological activity of FGF-20. As used herein, the term “biological activity” means that a CG53135 protein possesses some but not necessarily all the same properties of (and not necessarily to the same degree as) FGF-20.

A member (e.g., a protein and/or a nucleic acid encoding the protein) of the CG53135 family may further be given an identification name. For example, CG53135-01 (SEQ ID NOs:1 and 2) represents the first identified FGF-20 (see U.S. patent application Ser. No. 09/494,585); CG53135-05 (SEQ ID NOs:8 and 2) represents a codon-optimized, full length FGF-20 (i.e., the nucleic acid sequence encoding FGF-20 has been codon optimized, but the amino acid sequence has not been changed from the originally identified FGF-20); CG53135-12 (SEQ ID NOs:21 and 22) represent a single nucleotide polymorphism (“SNP”) of FGF-20 where one amino acid in CG53135-12 is different from SEQ ID NO:2 (the aspartic acid at position 206 is changed to asparagine, “²⁰⁶D→N”). Some members of the CG53135 family may differ in their nucleic acid sequences but encode the same CG53135 protein, e.g., CG53135-01, CG53135-03, and CG53135-05 all encode the same CG53135 protein. An identification name may also be an in-frame clone (“IFC”) number, for example, IFC 250059629 (SEQ ID NOs:33 and 34) represents amino acids 63-196 of the full length FGF-20 (cloned in frame in a vector). Table 1 shows a summary of some of the CG53135 family members. In one embodiment, the invention includes a variant of FGF-20 protein, in which some amino acids residues, e.g., no more than 1%, 2%, 3%, 5%, 10% or 15% of the amino acid sequence of FGF-20 (SEQ ID NO:2), are changed. In another embodiment, the invention includes nucleic acid molecules that can hybridize to FGF-20 under stringent hybridization conditions.

TABLE 1
Summary of some of the CG53135 family members

| | SEQ ID NO | |
|---|---|---|
| Name | (DNA/Protein) | Brief Description |
| CG53135-01 | 1 and 2 | FGF-20 wild type, stop codon removed |
| CG53135-02 | 3 and 4 | Codon optimized, amino acids 2-54 (as numbered in SEQ ID |
| | | NO: 2) were removed |
| CG53135-03 | 5 and 2 | FGF-20 wild type |
| CG53135-04 | 6 and 7 | Amino acids 20-51 (as numbered in SEQ ID NO: 2) were removed, |
| | | also valine at position 85 is changed to alanine (“⁸⁵V→A”) |
| CG53135-05 | 8 and 2 | Codon optimized, full length FGF-20 |
| CG53135-06 | 9 and 10 | Amino acids 20-51 (as numbered in SEQ ID NO: 2) were removed |
| CG53135-07 | 11 and 12 | Protein consisting of amino acids 1-18 (as numbered in SEQ ID |
| | | NO: 2) |
| CG53135-08 | 13 and 14 | Protein consisting of amino acids 32-52 (as numbered in SEQ ID |
| | | NO: 2) |
| CG53135-09 | 15 and 16 | Protein consisting of amino acids 173-183 (as numbered in SEQ |
| | | ID NO: 2) |
| CG53135-10 | 17 and 18 | Protein consisting of amino acids 192-211 (as numbered in SEQ |
| | | ID NO: 2) |
| CG53135-11 | 19 and 20 | Protein consisting of amino acids 121-137 (as numbered in SEQ |
| | | ID NO: 2) |
| CG53135-12 | 21 and 22 | FGF-20 SNP, aspartic acid at position 206 is changed to |
| | | asparagines (“²⁰⁶D→N”) as compared to CG53135-01 |
| CG53135-13 | 23 and 24 | CG53135-05 minus first 2 amino acids at the N-terminus |
| CG53135-14 | 25 and 26 | CG53135-05 minus first 8 amino acids at the N-terminus |
| CG53135-15 | 27 and 28 | CG53135-05 minus first 11 amino acids at the N-terminus |
| CG53135-16 | 29 and 30 | CG53135-05 minus first 14 amino acids at the N-terminus |
| CG53135-17 | 31 and 32 | CG53135-05 minus first 23 amino acids at the N-terminus |
| IFC 250059629 | 33 and 34 | In frame clone, open reading frame comprising a nucleotide |
| | | sequence encoding amino acids 63-196 of FGF-20 (SEQ ID NO: 2) |
| IFC 250059669 | 35 and 36 | In frame clone, open reading frame comprising a nucleotide |
| | | sequence encoding amino acids 63-211 of FGF-20 (SEQ ID NO: 2) |
| IFC 317459553 | 37 and 38 | In frame clone, open reading frame comprising a nucleotide |
| | | sequence encoding amino acids 63-194 of FGF-20 (SEQ ID NO: 2) |
| | | with ¹⁵⁹G→E |
| IFC 317459571 | 39 and 40 | In frame clone, open reading frame comprising a nucleotide |
| | | sequence encoding amino acids 63-194 of FGF-20 (SEQ ID NO: 2) |
| IFC 250059596 | 41 and 10 | In frame clone, open reading frame comprising a nucleotide |
| | | sequence encoding amino acids 1-19 and 52-211 of FGF-20 (SEQ |
| | | ID NO: 2) |
| IFC 316351224 | 41 and 10 | In frame clone, open reading frame comprising a nucleotide |
| | | sequence encoding amino acids 1-19 and 52-211 of FGF-20 (SEQ |
| | | ID NO: 2). |

As used herein, the term “effective amount” refers to the amount of a therapy (e.g., a composition comprising a CG53135 protein) which is sufficient to reduce and/or ameliorate the severity and/or duration of a disease (e.g., a joint disease, ischemic stroke, hemorrhagic stroke, trauma, spinal cord damage, heavy metal or toxin poisoning, or neurodegenerative diseases) or one or more symptoms thereof, prevent the advancement of a disease, cause regression of a disease, prevent the recurrence, development, or onset of one or more symptoms associated with a disease, or enhance or improve the prophylactic or therapeutic effect(s) of another therapy.

As used herein, the term “FGF-20” refers to a protein comprising an amino acid sequence of SEQ ID NO:2, or a nucleic acid sequence encoding such a protein or the complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the terms “prevent,” “preventing,” and “prevention” refer to the prevention of the recurrence, onset, or development of a disease (e.g., a joint disease, ischemic stroke, hemorrhagic stroke, trauma, spinal cord damage, heavy metal or toxin poisoning, or neurodegenerative diseases) or one or more symptoms thereof in a subject resulting from the administration of a therapy (e.g., a composition comprising a CG53135 protein), or the administration of a combination of therapies.

As used herein, the term “prophylactically effective amount” refers to the amount of a therapy (e.g., a composition comprising a CG53135 protein) which is sufficient to result in the prevention of the development, recurrence, or onset of a disease (e.g., a joint disease, ischemic stroke, hemorrhagic stroke, trauma, spinal cord damage, heavy metal or toxin poisoning, or neurodegenerative diseases) or one or more symptoms thereof, or to enhance or improve the prophylactic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. The term “subject” is used interchangeably with “patient” in the present invention.

As used herein, the terms “treat,” “treatment,” and “treating” refer to the reduction of the progression, severity, and/or duration of a disease (e.g., a joint disease, ischemic stroke, hemorrhagic stroke, trauma, spinal cord damage, heavy metal or toxin poisoning, or neurodegenerative diseases) or amelioration of one or more symptoms thereof, wherein such reduction and/or amelioration result from the administration of one or more therapies (e.g., a composition comprising a CG53135 protein).

As used herein, the term “therapeutically effective amount” refers to the amount of a therapy (e.g., a composition comprising a CG53135 protein), which is sufficient to reduce the severity of a disease (e.g., a joint disease, ischemic stroke, hemorrhagic stroke, trauma, spinal cord damage, heavy metal or toxin poisoning, or neurodegenerative diseases), reduce the duration of a disease, prevent the advancement of a disease, cause regression of a disease, ameliorate one or more symptoms associated with a disease, or enhance or improve the therapeutic effect(s) of another therapy.

### 4. BRIEF DESCRIPTION OF THE DRAWINGS

FIG. 1 shows Liquid Chromatography and Mass Spectrometry analysis of CG53135-05 E. coli purified product.

FIG. 2 depicts tryptic map of CG53135-05 E. coli purified product.

FIG. 3 shows Receptor Binding Specificity of CG53135. NIH 3T3 cells were serum-starved, incubated with the indicated factor (green squares=platelet derived growth factor; blue triangle=FGF-1; red circle=CG53135) either alone or together with the indicated soluble FGFR, and DNA synthesis in response to CG53135 was measured in a BrdU incorporation assay. Data points represent the average obtained from triplicate wells, and are represented as the percent BrdU incorporation relative to cells receiving factor alone.

FIG. 4 shows dose Response of CG53135-induced DNA synthesis in NIH 3T3 Fibroblasts. Serum starved NIH 3T3 cells were treated with purified CG53135-01 (CG53135 in figure), 10% serum or vehicle only (control). DNA synthesis was measured in triplicate for each sample, using a BrdU incorporation assay. Data points represent average BrdU incorporation and bars represent standard error (SE).

FIG. 5 shows CG53135 stimulates Growth of NIH 3T3 Fibroblasts. Duplicate wells of serum starved NIH 3T3 cells were treated for 1 day with purified CG53135-01 (1 ug) or vehicle control. Cell counts for each well were determined in duplicate. Y-axis identifies cell number, which is the average of 4 cell counts (treatment duplicates×duplicate counts) and standard error (SE).

FIG. 6 shows CG53135 induces DNA synthesis in 786-O Kidney Epithelial cells. Serum starved 786-O cells were left untreated or treated with partially purified CG53135-01 (from 5 ng/uL stock), or with vehicle control (mock). DNA synthesis was measured in triplicate for each sample, using a BrdU incorporation assay. Data points represent average BrdU incorporation and bars represent standard error (SE).

FIG. 7 shows the results of Forelimb Placing Test. The mean and standard error of the score for groups receiving vehicle (diamonds), 1.0 μg/injection CG53135-05 (square), and 2.5 μg/injection CG53135-05 (triangles) are represented over time. Asterisks indicate significant difference from vehicle control as assessed by one-way ANOVA.

FIG. 8 shows the results of Hindlimb Placing Test. The mean and standard error of the score for groups receiving vehicle (diamonds), 1.0 μg/injection CG53135 (square), and 2.5 μg/injection CG53135 (triangles) are represented over time. Asterisks indicate significant difference from vehicle control as assessed by one-way ANOVA.

FIG. 9 shows the results of Body Swing Test. The mean and standard error of the score for groups receiving vehicle (diamonds), 1.0 μg/injection CG53135 (square), and 2.5 μg/injection CG53135 (triangles) are represented over time. A score range of −50% swings to the right indicates no impairment, whereas 0% swings to the right swing indicates maximal impairment. Asterisks indicate significant difference from vehicle control as assessed by one-way ANOVA.

FIG. 10 shows the results of Cylinder Test. The mean and standard error of the score for groups receiving vehicle (diamonds), 1.0 μg/injection CG53135 (square), and 2.5 μg/injection CG53135 (triangles) are represented over time.

FIG. 11 shows the results of Body Weight. The mean and standard errors of the weights for groups receiving vehicle (diamonds), 1.0 μg/injection CG53135 (square), and 2.5 μg/injection CG53135 (triangles) is represented over time.

FIG. 12 shows the effect of CG53135 on Pro-MMP production in SW1353 cells in the presence of IL-1 beta.

FIG. 13 shows the effect of CG53135 on Pro-MMP production in SW1353 cells in the presence of TNF-alpha.

FIG. 14 shows the effect of CG53135 on TIMP production in SW1353 cells.

FIG. 15 shows the effect of intra-articular injection of CG53135 in the Meniscal Tear Model of Rat Osteoarthritis (Prophylactic Dosing): Mean Tibial Cartilage Degeneration.

FIG. 16 shows results of intra-articular injection of CG53135 in the Meniscal Tear Model of Rat Osteoarthritis: (Prophylactic Dosing): Total Cartilage Degeneration Width.

FIG. 17 shows results of intra-articular injection of CG53135 in the Meniscal Tear Model of Rat Osteoarthritis: (Prophylactic Dosing): Significant Tibial Cartilage Degeneration Width.

FIG. 18 shows results of intra-articular injection CG53135 in the Meniscal Tear Model of Rat Osteoarthritis (Therapeutic Dosing): Mean Tibial Degeneration.

FIG. 19 shows results of intra-articular injection of CG53135 in the Meniscal Tear Model of Rat Osteoarthritis (Therapeutic Dosing): Total Cartilage Degeneration Width.

FIG. 20 shows results of intra-articular injection of CG53135 in Meniscal Tear Model of Rat Osteoarthritis (therapeutic Dosing): Significant Tibial Cartilage Degeneration Width.

FIG. 21 (A) shows trophic action of EGF, NGF and CG53135; (B) shows the time course of CG53135-inhibited serum withdrawal-induced apoptosis.

FIG. 22 shows CG53135 inhibits serum withdrawal-induced caspase activation.

FIG. 23 shows neuritogenic action of CG53135 as compared to NGF.

FIG. 24 shows activation of MAPK by NGF and CG53135, and the inhibition of activity by PD98059, a MAPKK inhibitor.

### 5. DETAILED DESCRIPTION OF THE INVENTION

The present invention provides methods of preventing or treating a joint disease (e.g., osteoarthritis, other related joint pathologies, such as but are not limited to, ligament and tendon injuries within the ligament and tendon itself, or within their respective insertion sites, meniscal tears, other joint disorders where matrix deposition occurs, joint disorders where remodeling and repair are required, and cartilage and joint pathology occurred as a result of an inflammatory disease (e.g., rheumatoid arthritis)) in a subject comprising administering to the subject a composition comprising one or more CG53135 proteins.

The present invention also provides methods of using CG53135 to improve functional recovery following middle cerebral artery (MCA) occlusion. As stroke may result in disturbances of motor strength and coordination, sensory discrimination, visual function, speech, memory or other intellectual abilities, the present invention evaluates the efficacy and safety of CG53135 in a model that assesses these parameters. In accordance with the present invention, administering a composition comprising one or more CG53135 proteins is beneficial in the treatment of pathological conditions including, but are not limited to, ischemic stroke, hemorrhagic stroke, trauma, spinal cord damage, heavy metal or toxin poisoning, and neurodegenerative diseases (such as Alzheimer's disease, Parkinson's Disease, Amyotrophic Lateral Sclerosis, Huntington's Disease).

The present invention further provides fragments of FGF-20, e.g., CG53135-13 (SEQ ID NO:23 and 24), CG53135-14 (SEQ ID NO:25 and 26), CG53135-15 (SEQ ID NO:27 and 28), and CG53135-16 (SEQ ID NO:29 and 30), which possess the biological activities of the full length FGF-20.

For clarity of disclosure, and not by way of limitation, the detailed description of the invention is divided into the following subsections:
(i) CG53135
(ii) Methods of Preparing CG53135
(iii) Characterization and Demonstration of CG53.135 Activities and Monitoring Effects During Treatment
(iv) Prophylactic and Therapeutic Uses
(v) Pharmaceutical Compositions

5.1 CG53135

The present invention provides for compositions comprising CG53135 for prevention and/or treatment of a disease (e.g., a joint disease, ischemic stroke, hemorrhagic stroke, trauma, spinal cord damage, heavy metal or toxin poisoning, or neurodegenerative diseases). As used herein, the term “CG53135” refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs.

In one embodiment, a CG53135 protein is a variant of FGF-20. It will be appreciated by those skilled in the art that DNA sequence polymorphisms that lead to changes in the amino acid sequences of the FGF-20 protein may exist within a population (e.g., the human population). Such genetic polymorphism in the FGF-20 gene may exist among individuals within a population due to natural allelic variation. Such natural allelic variations can typically result in 1-5% variance in the nucleotide sequence of the FGF-20 gene. Any and all such nucleotide variations and resulting amino acid polymorphisms in the FGF-20 protein, which are the result of natural allelic variation of the FGF-20 protein, are intended to be within the scope of the invention. In one embodiment, a CG53135 is CG53135-12 (SEQ ID NOs:21 and 22), which is a single nucleotide polymorphism (“SNP”) of FGF-20 (i.e., ²⁰⁶D→N). (For more detailed description of CG53135-12, see e.g., U.S. patent application Ser. No. 10/702,126, filed Nov. 4, 2003, the disclosure of which is incorporated herein by reference in its entirety.) Additional examples of FGF-20 SNPs can be found in Example 2 of U.S. patent application Ser. No. 10/435,087, filed May 9, 2003, the content of which is incorporated by reference by its entirety.

In another embodiment, CG53135 refers to a nucleic acid molecule encoding a FGF-20 protein from other species or the protein encoded thereby, and thus has a nucleotide or amino acid sequence that differs from the human sequence of FGF-20. Nucleic acid molecules corresponding to natural allelic variants and homologues of the FGF-20 cDNAs of the invention can be isolated based on their homology to the human FGF-20 nucleic acids disclosed herein using the human cDNAs, or a portion thereof, as a hybridization probe according to standard hybridization techniques under stringent hybridization conditions.

In another embodiment, CG53135 refers to a fragment of an FGF-20 protein, including fragments of variant FGF-20 proteins, mature FGF-20 proteins, and variants of mature FGF-20 proteins, as well as FGF-20 proteins encoded by allelic variants and single nucleotide polymorphisms of FGF-20 nucleic acids. An example of an FGF-20 protein fragment includes, but is not limited to, residues 2-211, 3-211, 9-211, 12-211, 15-211, 24-211, 54-211, or 55-211 of FGF-20 (SEQ ID NO:2). In one embodiment, CG53135 refers to a nucleic acid encodes a protein fragment that includes residues 2-211, 3-211, 9-211, 12-211, 15-211, 24-211, 54-211, or 55-211 of SEQ ID NO:2.

The invention also encompasses derivatives and analogs of FGF-20. The production and use of derivatives and analogs related to FGF-20 are within the scope of the present invention.

In a specific embodiment, the derivative or analog is functionally active, i.e., capable of exhibiting one or more functional activities associated with a full-length, wild-type FGF-20. Derivatives or analogs of FGF-20 can be tested for the desired activity by procedures known in the art, including but not limited to, using appropriate cell lines, animal models, and clinical trials.

In particular, FGF-20 derivatives can be made via altering FGF-20 sequences by substitutions, insertions or deletions that provide for functionally equivalent molecules. In one embodiment, such alteration of an FGF-20 sequence is done in a region that is not conserved in the FGF protein family. Due to the degeneracy of nucleotide coding sequences, other DNA sequences which encode substantially the same amino acid sequence as FGF-20 may be used in the practice of the present invention. These include, but are not limited to, nucleic acid sequences comprising all or portions of FGF-20 that are altered by the substitution of different codons that encode a functionally equivalent amino acid residue within the sequence, thus producing a silent change. In a preferred embodiment, a wild-type FGF-20 nucleic acid sequence is codon-optimized to the nucleic acid sequence of SEQ ID NO:8 (CG53135-05). Likewise, the FGF-20 derivatives of the invention include, but are not limited to, those containing, as a primary amino acid sequence, all or part of the amino acid sequence of FGF-20 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-20 derivatives of the invention also include, but are not limited to, those containing, as a primary amino acid sequence, all or part of the amino acid sequence of FGF-20 including altered sequences in which amino acid residues are substituted for residues with similar chemical properties. In a specific embodiment, 1, 2, 3, 4, or 5 amino acids are substituted.

Derivatives or analogs of FGF-20 include, but are not limited to, those proteins which are substantially homologous to FGF-20 or fragments thereof, or whose encoding nucleic acid is capable of hybridizing to the FGF-20 nucleic acid sequence.

The FGF-20 derivatives and analogs of the invention can be produced by various methods known in the art. The manipulations that result in their production can occur at the gene or protein level. For example, the cloned FGF-20 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-20, care should be taken to ensure that the modified gene remains within the same translational reading frame as FGF-20, uninterrupted by translational stop signals, in the gene region where the desired FGF-20 activity is encoded.

Additionally, the FGF-20-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB® linkers (Pharmacia), etc.

Manipulations of the FGF-20 sequence may also be made at the protein level. Included within the scope of the invention are FGF-20 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2- groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-20 can be chemically synthesized. For example, a protein corresponding to a portion of FGF-20 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-20 sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the FGF-20 derivative is a chimeric or fusion protein comprising FGF-20 or a fragment thereof fused via a peptide bond at its amino- and/or carboxy-terminus to a non-FGF-20 amino acid sequence. In one embodiment, the non-FGF-20 amino acid sequence is fused at the amino-terminus of an FGF-20 or a fragment thereof. In another embodiment, such a chimeric protein is produced by recombinant expression of a nucleic acid encoding the protein (comprising an FGF-20-coding sequence joined in-frame to a non-FGF-20 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-20 with a heterologous signal sequence is expressed such that the chimeric protein is expressed and processed by the cell to the mature FGF-20 protein. The primary sequence of FGF-20 and non-FGF-20 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of FGF-20 molecule fused to a heterologous (non-FGF-20) protein-encoding sequence may be constructed. In a specific embodiment, such chimeric construction can be used to enhance one or more desired properties of an FGF-20, including but not limited to, FGF-20 stability, solubility, or resistance to proteases. In another embodiment, chimeric construction can be used to target FGF-20 to a specific site. In yet another embodiment, chimeric construction can be used to identify or purify an FGF-20 of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135 protein is carbamylated.

In some embodiment, a CG53135 protein can be modified so that it has an extended half-life in vivo using any methods known in the art. For example, Fc fragment of human IgG or inert polymer molecules such as high molecular weight polyethyleneglycol (PEG) can be attached to a CG53135 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135 protein. Unreacted PEG can be separated from CG53135-PEG conjugates by size-exclusion or by ion-exchange chromatography. PEG-derivatized conjugates can be tested for in vivo efficacy using methods known to those of skill in the art.

A CG53135 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG53135 refers to CG53135-01 (SEQ ID NOs:1 and 2), CG53135-02 (SEQ ID NOs:3 and 4), CG53135-03 (SEQ ID NOs:5 and 2), CG53135-04 (SEQ ID NOs:6 and 7), CG53135-05 (SEQ ID NOs:8 and 2), CG53135-06 (SEQ ID NOs:9 and 10), CG53135-07 (SEQ ID NOs:11 and 12), CG53135-08 (SEQ ID NOs:13 and 14), CG53135-09 (SEQ ID NOs:15 and 16), CG53135-10 (SEQ ID NOs:17 and 18), CG53135-11 (SEQ ID NOs:19 and 20), CG53135-12 (SEQ ID NOs:21 and 22), CG53135-13 (SEQ ID NOs:23 and 24), CG53135-14 (SEQ ID NOs:25 and 26), CG53135-15 (SEQ ID NOs:27 and 28), CG53135-16 (SEQ ID NOs:29 and 30), CG53135-17 (SEQ ID NOs:31 and 32), IFC 250059629 (SEQ ID NOs:33 and 34), IFC 20059669 (SEQ ID NOs:35 and 36), IFC 317459553 (SEQ ID NOs:37 and 38), IFC 317459571 (SEQ ID NOs:39 and 40), IFC 250059596 (SEQ ID NOs:41 and 10), IFC316351224 (SEQ ID NOs:41 and 10), or a combination thereof. In a specific embodiment, a CG53135 is carbamylated, for example, a carbamylated CG53135-13 protein or a carbamylated CG53135-05 protein.

5.2 Methods of Preparing CG53135

Methods of isolating a CG53135 protein are described in previous applications, e.g., U.S. patent application Ser. No. 09/609,543, filed Jul. 3, 2000, and Ser. No. 10/174,394, filed Jun. 17, 2002, both of which are incorporated herein by reference. Any techniques known in the art can be used in purifying a CG53135 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays. The purity of CG53135 can be assayed by any methods known in the art, such as but not limited to, gel electrophoresis. See Scopes, supra. In some embodiment, the CG53135 proteins employed in a composition of the invention can be in the range of 80 to 100 percent of the total mg protein, or at least 80%, at least 85%, at least 90%, at least 95%, or at least 98% of the total mg protein. In one embodiment, one or more CG53135 proteins employed in a composition of the invention is at least 99% of the total protein. In another embodiment, CG53135 is purified to apparent homogeneity, as assayed, e.g., by sodium dodecyl sulfate polyacrylamide gel electrophoresis.

Methods known in the art can be utilized to recombinantly produce CG53135 proteins. A nucleic acid sequence encoding a CG53135 protein can be inserted into an expression vector for propagation and expression in host cells.

An expression construct, as used herein, refers to a nucleic acid sequence encoding a CG53135 protein operably associated with one or more regulatory regions that enable expression of a CG53135 protein in an appropriate host cell. “Operably-associated” refers to an association in which the regulatory regions and the CG53135 sequence to be expressed are joined and positioned in such a way as to permit transcription, and ultimately, translation.

The regulatory regions that are necessary for transcription of CG53135 can be provided by the expression vector. A translation initiation codon (ATG) may also be provided if a CG53135 gene sequence lacking its cognate initiation codon is to be expressed. In a compatible host-construct system, cellular transcriptional factors, such as RNA polymerase, will bind to the regulatory regions on the expression construct to effect transcription of the modified CG53135 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135 gene sequence or to insert a CG53135 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135 sequence operably associated with regulatory regions can be directly introduced into appropriate host cells for expression and production of a CG53135 protein without further cloning. See, e.g., U.S. Pat. No. 5,580,859. The expression constructs can also contain DNA sequences that facilitate integration of a CG53135 sequence into the genome of the host cell, e.g., via homologous recombination. In this instance, it is not necessary to employ an expression vector comprising a replication origin suitable for appropriate host cells in order to propagate and express CG53135 in the host cells.

A variety of expression vectors may be used, including but are not limited to, plasmids, cosmids, phage, phagemids or modified viruses. Such host-expression systems represent vehicles by which the coding sequences of a CG53135 gene may be produced and subsequently purified, but also represent cells which may, when transformed or transfected with the appropriate nucleotide coding sequences, express CG53135 in situ. These include, but are not limited to, microorganisms such as bacteria (e.g., E. coli and B. subtilis) transformed with recombinant bacteriophage DNA, plasmid DNA or cosmid DNA expression vectors containing CG53135 coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135 coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135 molecule. For example, mammalian cells such as Chinese hamster ovary cells (CHO) can be used with a vector bearing promoter element from major intermediate early gene of cytomegalovirus for effective expression of a CG53135 sequence (Foecking et al., 1986, Gene 45:101; and Cockett et al., 1990, Bio/Technology 8:2).

In bacterial systems, a number of expression vectors may be advantageously selected depending upon the use intended for the CG53135 molecule being expressed. For example, when a large quantity of a CG53135 is to be produced, for the generation of pharmaceutical compositions of a CG53135 molecule, vectors that direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific proteases like enterokinase allows one to cleave off the CG53135 protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG53135 in infected hosts (see, e.g., Logan & Shenk, 1984, Proc. Natl. Acad. Sci. USA 8 1:355-359). Specific initiation signals may also be required for efficient translation of inserted CG53135 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, HeLa, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. Expression in a bacterial or yeast system can be used if post-translational modifications are found to be non-essential for a desired activity of CG53135. In a preferred embodiment, E. coli is used to express a CG53135 sequence.

For long-term, high-yield production of properly processed CG53135, stable expression in cells is preferred. Cell lines that stably express CG53135 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG53135 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk−, hgprt− or aprt− cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG53135. Modified culture conditions and media may also be used to enhance production of CG53135. Any techniques known in the art may be applied to establish the optimal conditions for producing CG53135.

An alternative to producing CG53135 or a fragment thereof by recombinant techniques is peptide synthesis. For example, an entire CG53135, or a protein corresponding to a portion of CG53135, can be synthesized by use of a peptide synthesizer. Conventional peptide synthesis or other synthetic protocols well known in the art may be used.

Proteins having the amino acid sequence of CG53135 or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135 protein is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG53135 can be found in Section 6, infra.

5.3 Characterization and Demonstration of CG53135 Activities and Monitoring Effects During Treatment

Any methods known in the art can be used to determine the identity of a purified CG53135 protein in a composition used in accordance to the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG53135 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure

The purity of a CG53135 protein in a composition used in accordance to the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG53135 protein in a composition used in accordance to the instant invention is at least 97%, at least 98%, or at least 99% pure by densitometry. In another preferred embodiment, a CG53135 protein in a composition used in accordance to the instant invention is more than 97%, more than 98%, or more than 99% pure by densitometry.

The biological activities and/or potency of CG53135 used in accordance with the present invention can be determined by any methods known in the art. For example, compositions for use in therapy in accordance to the methods of the present invention can be tested in suitable cell lines for one or more activities that FGF-20 possesses (e.g., cellular proliferation stimulatory activity). Non-limiting examples of such assays are described in Section 6.5, infra.

Compositions for use in a therapy in accordance to the methods of the present invention can also be tested in suitable animal model systems prior to testing in humans. Such animal model systems include, but are not limited to, rats, mice, hamsters, chicken, cows, monkeys, rabbits, etc. To establish an estimate of drug activity in animal model experiments, an index can be developed that combines observational examination of the animals as well as their survival status.

Further, any assays known to those skilled in the art can be used to evaluate the prophylactic and/or therapeutic utilities of the combinatorial therapies disclosed herein for prevention and/or treatment of a disease (e.g., a joint disease, ischemic stroke, hemorrhagic stroke, trauma, spinal cord damage, heavy metal or toxin poisoning, or neurodegenerative diseases).

The effectiveness of CG53135 on preventing and/or treating a disease (e.g., a joint disease, ischemic stroke, hemorrhagic stroke, trauma, spinal cord damage, heavy metal or toxin poisoning, or neurodegenerative diseases) can be monitored by any methods known to one skilled in the art, including but not limited to, clinical evaluation, and measuring the level of CG53135 biomarkers in a biosample. CG53135 biomarkers include, but are not limited to, CXCL1, IL-6, IL-8.

Any adverse effects during the use of CG53135 alone or in combination with another therapy (e.g., another therapeutic or prophylactic agent) are preferably also monitored. Examples of adverse effects of administering a CG53135 protein include, but are not limited to, nausea; chills; fever; vomiting; dizziness; photopsia (vision-“lights flashing”) and astigmatism (mild astigmatism); neuropathy (on soles of the feet); tachycardia; headache; and asymptomatic, and single premature atrial complex noted on ECG. Examples of adverse effects of other chemotherapies may be found in the Physicians' Desk Reference (58th ed., 2004).

5.4 Prophylactic and Therapeutic Uses

The present invention provides methods of preventing and/or treating a disease (e.g., a joint disease, ischemic stroke, hemorrhagic stroke, trauma, spinal cord damage, heavy metal or toxin poisoning, or neurodegenerative diseases) comprising administering to a subject in need thereof an effective amount of a composition comprising one or more isolated CG53135 proteins.

In one embodiment, the present invention provides methods of preventing and/or treating arthritis (e.g., osteoarthritis or rheumatic arthritis) comprising administering to a subject in need thereof a composition comprising one or more CG53135 proteins.

In another embodiment, the present invention provides methods of reducing cartilage degeneration comprising administering to a subject in need thereof a composition comprising one or more CG53135 proteins. In another embodiment, the present invention provides methods of stimulating cartilage repair comprising administering to a subject in need thereof a composition comprising one or more CG53135 proteins. In a specific embodiment, the present invention provides methods of stimulating cartilage healing after surgery in a subject comprising administering to a subject a composition comprising one or more CG53135 proteins.

In another embodiment, the present invention provides methods of preventing and/or treating a cardiovascular disease, such as stroke (e.g., ischemic stroke, hemorrhagic stroke), comprising administering to a subject a composition comprising one or more CG53135 proteins. In a specific embodiment, the present invention provides methods of preventing and/or treating a cardiovascular disease, such as stroke, comprising administering to a subject a composition comprising an isolated protein comprising an amino acid sequence of SEQ ID NO: 4,7,10,22, 24, 26, 28, 30, 32, 34, 36, 38, or 40.

In another embodiment, the present invention provides methods of preventing and/or treating a neurodegenerative disease (e.g., Alzheimer's disease, Parkinson's disease, Amyotrophic Lateral Sclerosis, Huntington's disease) comprising administering to a subject in need thereof a composition comprising one or more CG53135 proteins. In a specific embodiment, the present invention provides methods of preventing and/or treating a neurodegenerative disease comprising administering to a subject in need thereof a composition comprising an isolated protein comprising an amino acid sequence of SEQ ID NO: 4, 7, 10, 22, 24, 26, 28, 30, 32, 34, 36, 38, or 40.

In some embodiments, the present invention provides a method of preventing and/or treating a disease (e.g., a joint disease, ischemic stroke, hemorrhagic stroke, trauma, spinal cord damage, heavy metal or toxin poisoning, or neurodegenerative diseases) comprising cyclically administering a composition comprising one or more CG53135 proteins. In one embodiment, cycling therapy involves the administration of a first therapy for a period of time, followed by the administration of a second therapy for a period of time and repeating this sequential administration, i.e., the cycle, in order to, e.g., to avoid or reduce the side effects of one of the therapies and/or to improve the efficacy of the therapies. In another embodiment, cycling therapy involves the administration of a therapy for a period of time, stop the therapy for a period of time, and repeat the administration of the therapy. In accordance to the present invention, a composition comprising one or more CG53135 proteins can be administered to a subject prior to, during, or after the administration of a radiation therapy and/or chemotherapy, where such radiation therapy and/or chemotherapy is a cycling therapy.

In accordance to the instant invention, a composition comprising one or more isolated CG53135 proteins can also be used in combination with other therapies to prevent and/or treat a disease (e.g., a joint disease, ischemic stroke, hemorrhagic stroke, trauma, spinal cord damage, heavy metal or toxin poisoning, or neurodegenerative diseases). In one embodiment, a composition comprising one or more isolated CG53135 proteins is administered in combination with one or more other agents that have prophylactic and/or therapeutic effect(s) on a disease (e.g., a joint disease, ischemic stroke, hemorrhagic stroke, trauma, spinal cord damage, heavy metal or toxin poisoning, or neurodegenerative diseases) and/or have amelioration effect(s) on one or more symptoms associated with the disease to a subject to prevent and/or treat the disease. Any other agents or therapies that are known in the art that can be used to prevent and/or treat a disease, such as a joint disease, ischemic stroke, hemorrhagic stroke, trauma, spinal cord damage, heavy metal or toxin poisoning, or neurodegenerative diseases, can be used in combination with a composition comprising one or more CG53135 proteins in accordance to the methods of the present invention. In a specific embodiment, the present invention provides methods of stimulating cartilage healing after surgery in a subject comprising administering to a subject a composition comprising one or more CG53135 proteins.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more G53135 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg (as measured by UV assay). In another embodiment, the dosage of a composition comprising one or more CG53135 proteins for administration in a human patient provided by the present invention is between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG53135 protein (instead of IgG). Test results obtained with this UV method (using CG53135 reference standard) are three times lower than test results for the same sample(s) tested with the Bradford method (using IgG as calibrator). For example, if a dosage of a composition comprising one or more CG53135 proteins for administration in a human patient provided by the present invention is between 0.001-10 mg/kg measured by UV assay, then the dosage is 0.003-30 mg/kg as measured by Bradford assay.

In one embodiment, prior to administering the first full dose, each patient preferably receives a subcutaneous injection of a small amount (e.g., 1/100 to 1/10 of the prescribed dose) of a composition of the invention to detect any acute intolerance. The injection site is examined one and two hours after the test. If no reaction is detected, then the full dose is administered.

5.5 Pharmaceutical Compositions

The compositions of the invention can be administered to a subject at a prophylactically or therapeutically effective amount to prevent and/or treat a disease (e.g., a joint disease, ischemic stroke, hemorrhagic stroke, trauma, spinal cord damage, heavy metal or toxin poisoning, or neurodegenerative diseases). Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, nasal mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG53135 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in various salt forms, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG53135 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG53135 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG53135 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG53135 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising CG53135 may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG53135 can be formulated in an aerosol form, spray, mist or in the form of drops or powder if intranasal administration is preferred. In one embodiment, where a composition comprising one or more CG53135 proteins is desirable to be delivered to the brain (e.g., in the case of treating or preventing a neurodegenerative disease or stroke), nasal delivery of the composition is used. In particular, a composition comprising CG53135 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135 proteins may also be formulated into a microcapsule with one or more polymers (e.g., hydroxyethyl starch) form the surface of the microcapsule. Such formulations have benefits such as slow-release.

A composition comprising CG53135 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine; polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG53135 can be delivered to a subject by pulmonary administration, e.g., by use of an inhaler or nebulizer, of a composition formulated with an aerosolizing agent.

In a preferred embodiment, a composition comprising CG53135 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG53135 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG53135 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M-0.2 M acetate, 0.5-5% glycerol, 0.2-0.5 M arginine-HCl, and one or more CG53135 proteins, preferably 0.5-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG53135 proteins, preferably 0.8 mg/ml (UV). In some embodiments, a formulation used in accordance to the methods of the invention comprises 0.01-1 M of a stabilizer, such as arginine in various salt forms, sulfobutyl ether Beta-cyclodextrin sodium, or sucrose, 0.01-0.1 M sodium phosphate monobasic (NaH₂PO₄.H₂O), 0.01%-0.1% weight/volume (“w/v”) polysorbate 80 or polysorbate 20, and one or more CG53135 proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG53135 proteins.

The invention also provides kits for carrying out the therapeutic regimens of the invention. Such kits comprise in one or more containers prophylactically or therapeutically effective amounts of the composition of the invention (e.g., a composition comprising one or more CG53135 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG53135 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### 6. EXAMPLE

Certain embodiments of the invention are illustrated by the following non-limiting examples.

### 6.1 Example 1

### Identification of Single Nucleotide Polymorphisms in FGF-20 Nucleic Acid Sequences

This example demonstrated how some of the single nucleotide polymorphisms (SNPs) of FGF-20 were identified. A SNP can, in some instances, be referred to as a “cSNP” to denote that the nucleotide sequence containing the SNP originates as a cDNA. SNPs occurring within a gene may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Non-limiting examples include alteration in temporal expression, physiological response regulation, cell type expression regulation, intensity of expression, and stability of transcribed message.

SeqCalling™ assemblies produced by the exon linking process were selected and extended using the following criteria: genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling™ assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling™ assemblies map to those regions. Such SeqCalling™ sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling™ database. SeqCalling™ fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling™ assemblies and genomic clones was reiterated to derive the full length sequence (Alderborn et al., Genome Research 10 (8) 1249-1265 (2000)).

Variants are reported individually in Table 2, but any combination of all or select subset of the variants is also encompassed by the present invention.

TABLE 2
SNPs of CG53135-01 (SEQ ID NOs: 1 and 2)

| | Nucleotides | | | Amino Acids | | |
|---|---|---|---|---|---|---|
| Variant | Position | Initial | Modified | Position | Initial | Modified |
| 13377871 | 301 | A | G | 101 | Ile | Val |
| 13375519 | 361 | A | G | 121 | Met | Val |
| 13375518 | 517 | G | A | 173 | Gly | Arg |
| 13375516 | 523 | C | G | 175 | Pro | Ala |
| 13381791 | 616 | G | A | 206 | Asp | Asn |

### 6.2 Example 2

### Expression of CG53135

Several different expression constructs were generated to express CG53135 proteins (Table 3). The CG53135-05 construct, a codon-optimized, phage-free construct encoding the full-length gene (construct #3 in Table 3), was expressed in E. coli BLR (DE3), and the purified protein product was used in toxicology studies and clinical trials.

TABLE 3
Constructs Generated to Express CG53135

| Construct | Construct Description | Construct Diagram |
|---|---|---|
| 1a | NIH 3T3 cells were transfected with pFGF-20,which incorporates an epitope tag (V5) and apolyhistidine tag into the carboxy-terminus ofthe CG53135-01 protein in the pcDNA3.1vector (Invitrogen) | 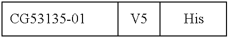 |
| 1b | Human 293-EBNA embryonic kidney cells orNIH 3T3 cells were transfected with CG53135-01 using pCEP4 vector (Invitrogen) containing an IgK signal sequence, multiple cloning sites,a V5 epitope tag, and a polyhistidine tag | 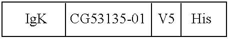 |
| 2 | E. coli BL21 cells were transformed withCG53135-01 using pETMY vector (CuraGenCorporation) containing a polyhistidine tag anda T7 epitope tag (this construct is also referredto as E. coli/pRSET) | 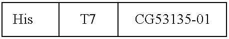 |
| 3 | E. coli BLR (DE3) cells (NovaGen) weretransformed with CG53135-05 (full-length,codon-optimized) using pET24a vector(NovaGen) | 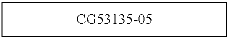 |
| 4 | E. coli BLR (DE3) cells (NovaGen) weretransformed with CG53135 (deletion of aminoacids 2-54, codon-optimized) using pET24avector (NovaGen) | 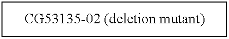 |

In one construct, CG53135-01 (the full-length CG53135 gene) was cloned as a Bgl II-Xho I fragment into the Bam HI-Xho I sites in mammalian expression vector, pcDNA3.1V5His (Invitrogen Corporation, Carlsbad, Calif.). The resultant construct, pFGF-20 (construct 1a) has a 9 amino acid V5 tag and a 6 amino acid histidine tag (His) fused in-frame to the carboxy-terminus of CG53135-01. These tags aid in the purification and detection of CG53135-01 protein. After transfection of pFGF-20 into murine NIH 3T3 cells, CG53135-01 protein was detected in the conditioned medium using an anti-V5 antibody (Invitrogen, Carlsbad, Calif.).

The full-length CG53135-01 gene was also cloned as a Bgl II-Xho I fragment into the Bam HI-Xho I sites of mammalian expression vector pCEP4/Sec (CuraGen Corporation). The resultant construct, pIgK-FGF-20 (construct 1b) has a heterologous immunoglobulin kappa (IgK) signal sequence that could aid in secretion of CG53135-01. After transfection of pIgK-FGF-20 into human 293 EBNA cells (Invitrogen, Carlsbad, Calif.; catalog #R620-07), CG53135-01 was detected in the conditioned medium using an anti-V5 antibody.

In order to increase the yield of CG53135 protein, a Bgl II-Xho I fragment encoding the full-length CG53135-01 gene was cloned into the Bam HI-Xho I sites of E. coli expression vector, pETMY (CuraGen Corporation). The resultant construct, pETMY-FGF-20 (construct 2) has a 6 amino acid histidine tag and a T7 tag fused in-frame to the amino terminus of CG53135. After transformation of pETMY-FGF-20 into BL21 E. coli (Novagen, Madison, Wis.), followed by T7 RNA polymerase induction, CG53135-01 protein was detected in the soluble fraction of the cells.

In order to express CG53135 without tags, CG53135-05 (a codon-optimized, full-length FGF-20 gene) and CG53135-02 (a codon-optimized deletion construct of FGF-20, with the N-terminal amino acids 2-54 removed) were synthesized. For the full-length construct (CG53135-05), an Nde I restriction site (CATATG) containing the initiator codon was placed at the 5′ end of the coding sequence. At the 3′ end, the coding sequence was followed by 2 consecutive stop codons (TAA) and a Xho restriction site (CTCGAG). The synthesized gene was cloned into pCRScript (Stratagene, La Jolla, Calif.) to generate pCRScript-CG53135. An Nde I-Xho I fragment containing the codon-optimized CG53135 gene was isolated from the pCRscript-CG53135 and subcloned into Nde I-Xho 1-digested pET24a to generate pET24a-CG53135 (construct 3). The full-length, codon-optimized version of CG53135 is referred to as CG53135-05.

To generate a codon-optimized deletion construct for CG53135, oligonucleotide primers were designed to amplify the deleted CG53135 gene from pCRScript-CG53135. The forward primer contained an Nde I site (CATATG) followed by coding sequence starting at amino acid 55. The reverse primer contained a HindIII restriction site. A single PCR product of approximately 480 base pairs was obtained and cloned into pCR2.1 vector (Invitrogen) to generate pCR2.1-CG53135del. An Nde I-Hind III fragment was isolated from pCR2.1-53135del and subcloned into Nde I-Hind III-digested pET24a to generate pET24a-CG53135-02 (construct 4).

The plasmids, pET24a-CG53135-05 (construct 3) and pET24a-CG53135-02 (construct 4) have no tags. Each vector was transformed into E. coli BLR (DE3), induced with isopropyl thiogalactopyranoside. Both the full-length and the N-terminally truncated CG53135 protein was detected in the soluble fraction of cells.

### 6.3 Example 3

### Proteolytic Cleavage Products of CG53135-05

When pET24a-CG53135-05 (construct 3, see Example 2) was expressed in E. coli (DE3) and the protein was purified according to Process 1 as described in Section 6.17.1 and Process 2 as described in Section 6.17.2, respectively, the final purified protein product from each process was analyzed using techniques such as Liquid Chromatography, Mass spectrometry and N-terminal sequencing. Such analyses indicate that the final purified protein product includes some truncated form of FGF-20 (e.g., CG53135-13 (SEQ ID NO:24), CG53135-15 (SEQ ID NO:28), CG53135-16 (SEQ ID NO:30), and CG53135-17 (SEQ ID NO:32)) in addition to the full length FGF-20, and a protein consisting of amino acids 3-211 (CG53135-13, SEQ ID NO:24) of FGF-20 constitutes the majority of the final purified protein product.

All the variants/fragments in the final purified product have high activity in the proliferation assays. Thus these variants/fragments are expected to have same utility as that of FGF-20. For the purpose of convenience, the term “CG53135-05 E. coli purified product” is used herein to refer to a purified protein product from E. coli expressing a CG53135-05 construct. For example, a CG53135-05 E. coli purified product may contain a mixture of the full length CG53135-05 protein (SEQ ID NO:2), CG53135-13 (SEQ ID NO:24), CG53135-15 (SEQ ID NO:28), CG53135-16 (SEQ ID NO:30), and CG53135-17 (SEQ ID NO:32), with the majority of the content being CG53135-13 (SEQ ID NO:24).

### RP-HPLC Assay: Peak Identification

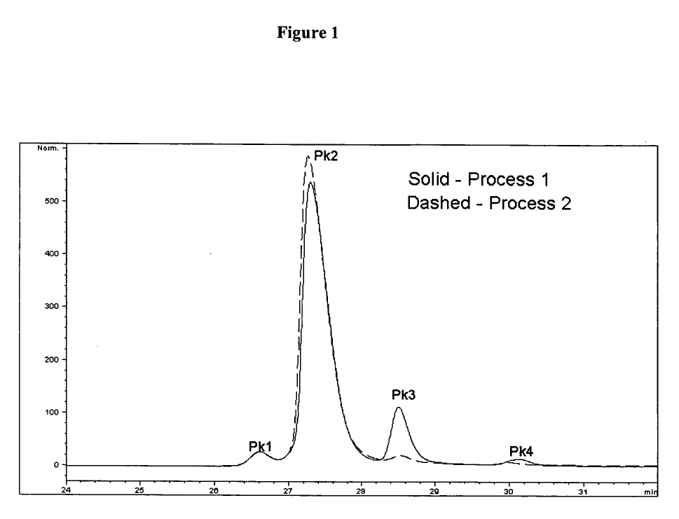

Purified drug substance (by both Process 1 and Process 2, respectively) was further analyzed by reversed-phase high-performance liquid chromatography (RP-HPLC) with both UV and electrospray mass spectrometric detection. Purified protein from either Process 1 or Process 2 was loaded onto a Protein C4 column (Vydac, 5 μm, 150 mm×4.6 mm) using a standard HPLC system in a mobile phase containing water, acetonitrile and trifluoroacetic acid. The elution gradient for this method was modified to resolve four distinct chromatographic peaks eluting at 26.6, 27.3, 28.5 and 30.0 min respectively (FIG. 1). These peaks were characterized by electrospray mass spectrometry. As can be observed from the chromatograms, the four equipotent isoforms are present in the purified final product from Process 1 and 2. However, the proportion of these peaks (1, 3 and 4) is much lower in the final product purified by Process 2 with the predominant form being Peak 2.

The identities of each peak from the RP-HPLC separation are indicated in Table 4.

TABLE 4
Identity of peaks from the RP-HPLC separation of CG53135-05
E. coli purified product based upon accurate molecular weight determination.

| | | Molecular | | | Predicted |
|---|---|---|---|---|---|
| | Retention | Weight | | | Molecular |
| Peak # | Time (min) | Observed | Assignment (residue #) | ID Number | Weight |
| 1 | 26.6 | 21329.2 | 24-211 | CG53135-17 | 21329.2 |
| 1 | 26.6 | 22185.1 | 15-211 | CG53135-16 | 22185.1 |
| 1 | 26.6 | 22412.4 | 12-211 | CG53135-15 | 22412.4 |
| 2 | 27.3 | 23296.5 | 3-211 | CG53135-13 | 23296.4 |
| 3 | 28.5 | 23498.9 | 1-211 | CG53135-05 | 23498.7 |
| 4 | 30.0 | 23339.3 | 3-211 (carbamylated) | CG53135-13 | 23339.4 |
| | | | | (carbamylated) | |
| 4 | 30.0 | 23539.7 | 1-211 (carbamylated) | CG53135-05 | 23539.7 |
| | | | | (carbamylated) | |

### Edman Sequencing and Total Amino Acid Analysis

The experimental N-terminal amino acid sequence of the Process 1 reference standard, DEV10, and the Process 2 interim reference standard were determined qualitatively. The reference standards were resolved by SDS-PAGE and electrophoretically transferred to a polyvinylidenefluoride membrane; the Coomassie-stained ˜23 kDa major band corresponding to each reference standard was excised from the membrane and analyzed by an automated Edman sequencer (Procise, Applied Biosystems, Foster City, Calif.). A comparison of the two major sequences is shown in Table 5 below. The predominant sequence for each reference standard was identical and corresponded to residues 3-20 in the theoretical N-terminal sequence of CG53135-05.

TABLE 5
Edman sequencing data for the first 20 amino acids of CG53135-05
E. coli purified product for Process 1 and 2.

| | |
|---|---|
| Theoretical | |
| Residue | Amino Acid Residue |

| Position | Process 1 | Process 2 |
|---|---|---|
| 3 | Pro | Pro |
| 4 | Leu | Leu |
| 5 | Ala | Ala |
| 6 | Glu | Glu |
| 7 | Val | Val |
| 8 | Gly | Gly |
| 9 | Gly | Gly |
| 10 | Phe | Phe |
| 11 | Leu | Leu |
| 12 | Gly | Gly |
| 13 | Gly | Gly |
| 14 | Leu | Leu |
| 15 | Glu | Glu |
| 16 | Gly | Gly |
| 17 | Leu | Leu |
| 18 | Gly | Gly |
| 19 | Gln | Gln |
| 20 | Gln | Gln |

The experimental amino acid composition of the DEV10 reference standard and the PX3536G001-H reference standard were determined in parallel. Quadruplicate samples of each reference standard were hydrolyzed for 16 hours at 115° C. in 100 μL of 6 N HCl, 0.2% phenol containing 2 nmol norleucine as an internal standard. Samples were dried in a Speed Vac Concentrator and dissolved in 100 μL sample buffer containing 2 nmol homoserine as an internal standard. The amino acids in each sample were separated on a Beckman Model 7300 amino acid analyzer. The amino acid composition of both reference standards showed no significant differences as shown in Table 6 below. Note that Cys and trp are destroyed during acid hydrolysis of the protein. Asn and gin are converted to asp and glu, respectively, during acid hydrolysis and thus their respective totals are reported as asx and glx. Met and his were both unresolved in this procedure.

TABLE 6
Quantitive amino acid analysis for CG53135-05 E. coli
purified product from Process 1 and Process 2

| | |
|---|---|
| Amino Acid | Mole Percent |

| Residue | DEV10 | PX3536G001-H |
|---|---|---|
| asx | 7.1 | 7.0 |
| thr | 4.0 | 4.0 |
| ser | 6.3 | 6.1 |
| glx | 12.2 | 12.2 |
| pro | 6.0 | 6.0 |
| gly | 14.4 | 14.3 |
| ala | 5.8 | 5.6 |
| val | 5.3 | 5.3 |
| ile | 3.5 | 3.5 |
| leu | 13.6 | 13.6 |
| tyr | 4.6 | 4.6 |
| phe | 5.2 | 5.2 |
| lys | 3.7 | 3.7 |
| arg | 8.5 | 9.1 |

### Tryptic Mapping by RP-HPLC

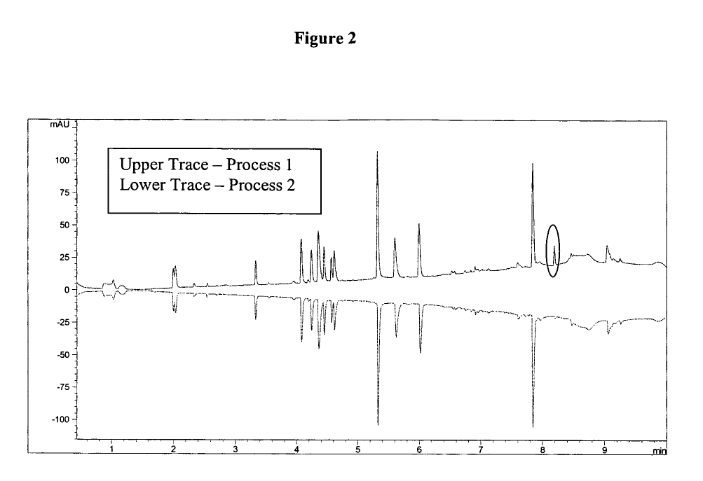

Purified drug substance from Process 1 and 2 was reduced and alklated with iodoacetic acid and then digested with sequencing grade trypsin. The tryptic peptides were separated by reversed-phase high-performance liquid chromatography (RP-HPLC) using both UV and electrospray mass spectrometric detection. The tryptic digest from either Process 1 or Process 2 was loaded onto an ODS-1 nonporous silica column (Micra, 1.5 μm; 53×4.6 mm) using a standard HPLC system in a mobile phase containing water, acetonitrile and trifluoroacetic acid. The eluting peptides were detected by UV at 214 nm (FIG. 2) and by positive-ion electrospray mass spectrometry. The major difference between the two chromatograms for Process 1 and Process 2 is the reduction in peak area of a peak obvious in the Process I trace (peak at 8.2 min; FIG. 2). This peak corresponds to the T1 peptide, residues 1-40. This observation is expected since the source of this peptide if from the intact CG53135-05, which is in greater abundance in the Process I material (peak 3, FIG. 1).

### Bioassay

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 hours. CG53135-05-related species were added and incubated for 18 hours at 37° C. in 10% CO₂/air. BrdU (10 mM final concentration) was added and incubated with the cells for 2 hours at 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was not included in this assay since insufficient material was collected (Peak 4 is less than 3% of the total peak area for CG53135-05). CG53135-05 and material collected from all 3 remaining fractions (i.e., Peak 1, 2, and 3) induced DNA synthesis in NIH 3T3 mouse fibroblasts in a dose-dependent manner (Table 7). The PI₂₀₀ was defined as the concentration of protein that resulted in incorporation of BrdU at 2 times the background. CG53135-05 and CG53135-05 related species recovered from all 3 measurable peaks demonstrated similar biological activity with a PI₂₀₀ of 0.7-11 ng/mL (Table 7).

TABLE 7
Biological Activity of CG53135-05 E. coli purified
product (DEV10): Induction of DNA Synthesis

| | CG53135-05 (DEV 10) | Peak 1 | Peak 2 | Peak 3 |
|---|---|---|---|---|
| PI₂₀₀ (ng/mL) | 1.0 | 0.7 | 11 | 8.6 |

### 6.4 Example 4

### Receptor Binding Specificity of CG53135 (Study L-116.01)

FGF family members transduce signals intracellularly via high affinity interactions with cell surface immunoglobulin (Ig) domain-containing tyrosine kinase FGF receptors (FGFRs). Four distinct human genes encode FGFRs (Powers et al., Endocr Relat Cancer 2000, 7:165-97; Klint and Claesson-Welsh, Front Biosci 1999, 4:D165-77; Xu et al., Cell Tissue Res 1999, 296:33-43). A related fifth human sequence lacking a kinase domain has recently been identified and named FGFR-5 (Kim et al., Biochim Biophys Acta 2001, 1518:152-6). These receptors can each bind several different members of this family (Kim et al., Biochim Biophys Acta 2001, 1518:152-6; Ornitz et al., J Biol Chem 1996, 271:15292-7). FGFs also bind, albeit with low affinity, to heparin sulfate proteoglycans (HSPGs) present on most cell surfaces and extracellular matrices (ECM). Interactions between FGFs and HSPGs serve to stabilize FGF/FGFR interactions and to sequester FGF and protect it from degradation (Powers et al., Endocr Relat Cancer 2000, 7:165-97; Szebenyi and Fallon, Int Rev Cytol 1999, 185:45-106). Dimerization of FGF receptor monomers upon ligand binding is reported to be a requisite for activation of the kinase domains, leading to receptor trans-phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

FGFR-1, FGFR-2 and FGFR-3 each recognize FGF-1, FGF-2, FGF-4 and FGF-8. In addition, FGFR-1 & FGFR-2 bind FGF-3, FGF-5, FGF-6, FGF-10 and FGF-17 (Powers et al., Endocr Relat Cancer 2000, 7:165-97). Binding of various FGF ligands varies with each receptor splice form, thus allowing a wide repertoire of FGF-mediated signaling events through a limited number of receptor coding genes. Tissue-specific alternate splicing permits cells expressing a single FGFR gene to significantly diversify their biological response by generating distinct receptor isoforms that may exhibit different ligand specificity and function. FGFR-4, binds FGF-1, FGF-2, FGF-4, FGF-6, FGF-8 and FGF-9 but not FGF-3, FGF-5 or FGF-7. FGF-7, or keratinocyte growth factor-1 (KGF-1) is only recognized by FGFR-2, whereas FGF-9 binds to FGFR-2, FGFR-3 and FGFR-4. Receptor specificity of FGFs-11 to -19 is not well understood (Powers et al., Endocr Relat Cancer 2000, 7:165-97; Ornitz et al., J Biol Chem 1996, 271:15292-7).

Immunohistochemistry studies (Hughes, J Histochem Cytochem 1997, 45:1005-19) in normal human adult tissues from the major organ systems indicated that FGFR-1, FGFR-2 and FGFR-3 are widely expressed, suggesting an important functional role in tissue homeostasis. Protein expression patterns for tissue-specific isoforms have not yet been determined. FGFR-4 has a more limited expression pattern being notably absent from lung, oviduct, placenta, testis, prostate, thyroid, parathyroid, and sympathetic ganglia, tissues where all three other receptors are predominantly expressed (Hughes, J Histochem Cytochem 1997, 45:1005-19).

To determine the receptor binding specificity of CG53135, we examined the effect of soluble FGFRs on the induction of DNA synthesis in NIH 3T3 cells by recombinant CG53135-01 produced in E. coli.

### Materials and Methods

Protein Purification from Escherichia coli: For production in E. coli, plasmid pETMY-hFGF20X was transformed into the E. coli expression host BL21 (Novagen, Madison, Wis.) and the induction of protein CG53135 expression was carried out according to the manufacturer's instructions. pETMYhFGF20X/BL21 E. coli bacteria were grown in LB medium at 37° C. At an OD of 0.6, bacteriophage lambda (CE6) was added to a final multiplicity of infection of 5. The infected culture was further incubated at 27° C. for 3 hours. After induction, total cells were harvested, and proteins were analyzed by Western blotting using anti-HisGly antibody (Invitrogen). Cells were harvested by low-speed centrifugation (5000 rpm in a GS-3 rotor for 15 minutes at 4° C.), suspended in phosphate-buffered saline (PBS) containing 0.5M NaCl and 1M arginine, and disrupted with two passes through a microfluidizer. Cell debris was removed by low-speed centrifugation and the soluble protein fraction (supernatant) was clarified by filtration through a 0.2 micron low-protein binding membrane. The protein sample was then loaded onto a metal chelation column (pre-charged with nickel sulfate). The nickel column was washed with PBS/0.5M NaCl+1M L-arginine and bound proteins were eluted with a linear gradient of imidazole (0-0.5 M). Fractions containing CG53135 (100-150 mM imidazole) were pooled and dialyzed against 1×106 volumes of PBS pH 8.0 containing 1M L-arginine. The protein sample was stored at −80° C.

Receptor Specificity: NIH 3T3 cells were cultured in 96-well plates to approximately 100% confluence, washed and fed with DMEM without supplements (Life Technologies), and incubated for 24 h. Recombinant CG53135-01 or control protein was then added to the cells for 18 hours. Control proteins used were aFGF (positive control) and platelet derived growth factor-BB (PDGFBB) (negative control). To analyze the effect of soluble FGFRs on CG53135 activity, recombinant CG53135-01, aFGF, or PDGF-BB (final concentrations of 10, 5 and 3 ng/mL, respectively), were mixed with soluble receptors (final concentrations of 0.2, 1 and 5 ug/mL), and incubated for 30 min at 37° C. prior to addition to serum-starved NIH 3T3 cells. Factor concentrations represent the amount of ligand needed to generate a half maximal BrdU response in NIH 3T3 cells. Soluble FGFRs were Fc chimeras of the following receptor forms (FGFR1β(IIIc), FGFR2β(IIIb), FGFR2 [symbol] (IIIb), FGFR2 [symbol] (IIIc), FGFR3 [symbol] (IIIc), FGFR4) and were obtained from R&D Systems (Minneapolis, Minn.). The BrdU assay was performed according to the manufacturer's specifications (Roche Molecular Biochemicals, Indianapolis, Ind.) using a 4 h BrdU incorporation time.

### Results and Conclusions

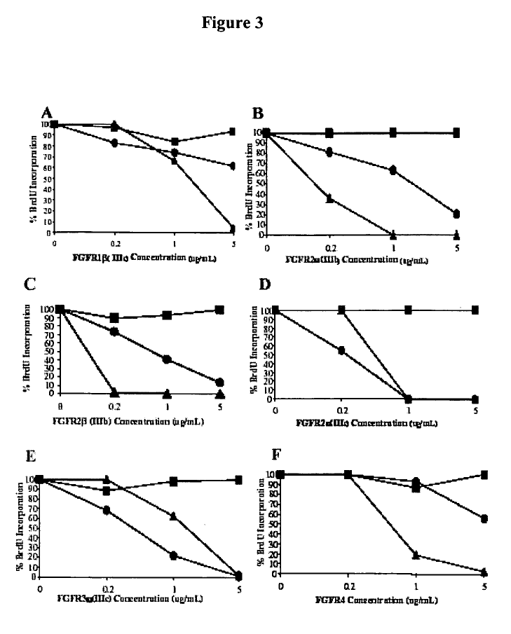

To determine the receptor binding specificity of CG53135, we examined the effect of soluble FGFRs on the induction of DNA synthesis in NIH 3T3 cells by recombinant CG53135-01 produced in E. coli. Soluble receptors for FGFR1β(IIIc), FGFR2β(IIIb), FGFR2 [symbol] (IIIb), FGFR2 [symbol] (IIIc), FGFR3 [symbol] (IIIc), and FGFR4 were utilized. We found that soluble forms of each of these FGFRs were able to specifically inhibit the biological activity of CG53135 (FIG. 3).

Complete or nearly complete inhibition was obtained with soluble FGFR2 [symbol] (IIIb), FGFR2β(IIIb), FGFR2 [symbol] (IIIc), and FGFR3 [symbol] (IIIc), whereas partial inhibition was achieved with soluble FGFR1β(IIIc) and FGFR4. None of the soluble receptor reagents interfered with the induction of DNA synthesis by PDGF-BB (FIG. 3), thereby demonstrating their specificity. The integrity of each soluble receptor reagent was demonstrated by showing their ability to inhibit the induction of DNA synthesis by aFGF, a factor known to interact with all of the FGFR's under analysis (FIG. 3).

### 6.5 Example 4

### Cellular Proliferation Responses with CG53135 (Studies L-117.01 and L-117.02)

Experiments were performed to evaluate the proliferative response of representative cell types to CG53135, e.g., a full-length tagged variant (CG53135-01), a deletion variant (CG53135-02), and a full-length codon-optimized untagged variant (CG53135-05).

### Materials and Methods:

Heterologous Protein Expression: CG53135-01 (batch 4A and 6) was used in these experiments. Protein was expressed using Escherichia coli (E. coli), BL21 (Novagen, Madison, Wis.), transformed with full-length CG53135-01 in a pETMY-hFGF20X/BL21 expression vector. Cells were harvested and disrupted, and then the soluble protein fraction was clarified by filtration and passed through a metal chelation column. The final protein fraction was dialyzed against phosphate buffered saline (PBS) plus 1 M L-arginine. Protein samples were stored at −70° C.

CG53135-02 (batch 1 and 13) was also used in these experiments. Protein was expressed in E. coli, BLR (DE3) (Novagen), transformed with the deletion variant CG53135-02 inserted into a pET24a vector (Novagen). A research cell bank (RCB) was produced and cell paste containing CG53135-02 was produced by fermentation of cells originating from the RCB. Cell membranes were disrupted by high-pressure homogenization, and lysate was clarified by centrifugation. CG53135-02 was purified by ion exchange chromatography. The final protein fraction was dialyzed against the formulation buffer (100 mM citrate, 1 mM ethylenediaminetetraacetic acid (EDTA), and 1 M L-arginine).

CG53135-05, DEV10, which were also used in these experiments, was prepared by Cambrex Biosciences (Hopkinton, Mass.) according to Process 1 as described in Section 6.14.1, infra.

BrdU Incorporation: proliferative activity was measured by treatment of serum-starved cultured cells with a given agent and measurement of BrdU incorporation during DNA synthesis. Cells were cultured in respective manufacturer recommended basal growth medium supplemented with 10% fetal bovine serum or 10% calf serum as per manufacturer recommendations. Cells were grown in 96-well plates to confluence at 37° C. in 10% CO₂/air (to subconfluence at 5% CO₂ for dedifferentiated chondrocytes and NHOst). Cells were then starved in respective basal growth medium for 24-72 hours. CG53135 protein purified from E. coli or pCEP4/Sec or pCEP4/Sec-FGF 20× enriched conditioned medium was added (10 μL/100 μL of culture) for 18 hours. BrdU (10 μM final concentration) was then added and incubated with the cells for 5 hours. BrdU incorporation was assayed according to the manufacturer's specifications (Roche Molecular Biochemicals, Indianapolis, Ind.).

Growth Assay: growth activity was obtained by measuring cell number following treatment of cultured cells with a given agent for a specified period of time. In general, cells grown to −20% confluency in 6-well dishes were treated with basal medium supplemented with CG53135 or control, incubated for several days, trypsinized and counted using a Coulter Z1 Particle Counter.

### Results:

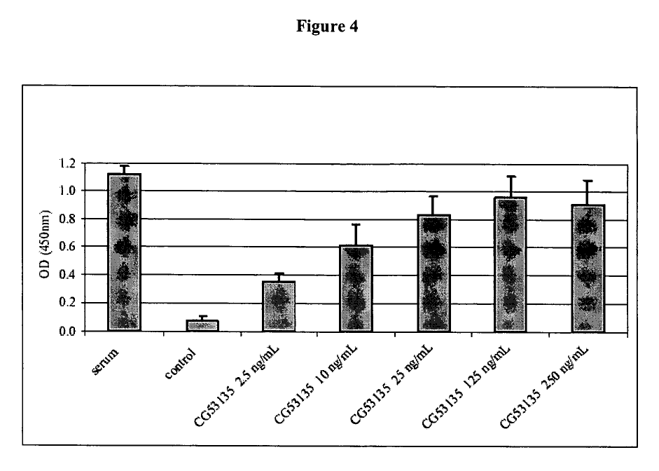

Proliferation in Mesenchymal Cells: to determine if recombinant CG53135 could stimulate DNA synthesis in fibroblasts, a BrdU incorporation assay was performed using CG53135-01 treated NIH 3T3 murine embryonic lung fibroblasts. Recombinant CG53135-01 induced DNA synthesis in NIH 3T3 mouse fibroblasts in a dose-dependent manner (FIG. 4). DNA synthesis was generally induced at a half maximal concentration of ˜10 ng/mL. In contrast, treatment with vehicle control purified from cells did not induce any DNA synthesis.

CG53135-01 also induced DNA synthesis in other cells of mesenchymal origin, including CCD-1070Sk normal human foreskin fibroblasts, MG-63 osteosarcoma cell line, and rabbit synoviocyte cell line, HIG-82. In contrast, CG53135-01 did not induce any significant increase in DNA synthesis in primary human osteoblasts (NHOst), human pulmonary artery smooth muscle cells, human coronary artery smooth muscle cells, human aorta smooth muscle cells (HSMC), or in mouse skeletal muscle cells.

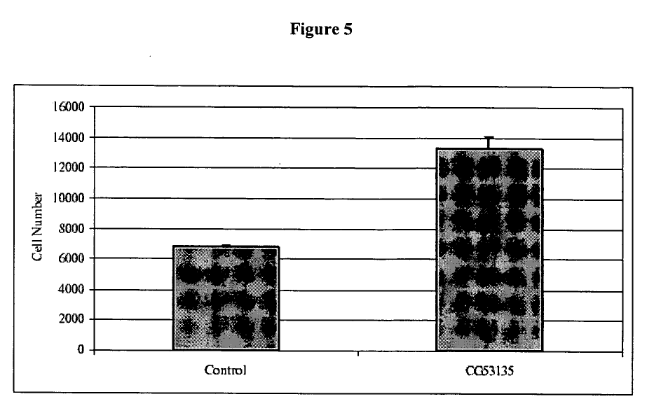

To determine if recombinant CG53135-01 sustained cell growth, NIH 3T3 cells were cultured with 1 μg CG53135-01 or control for 48 hours and then counted (FIG. 5). CG53135 induced an approximately 2-fold increase in cell number relative to control in this assay. These results show that CG53135 acts as a growth factor.

Proliferation of Epithelial Cells: to determine if recombinant CG53135 can stimulate DNA synthesis and sustain cell growth in epithelial cells, a BrdU incorporation assay was performed in representative epithelial cell lines treated with CG53135. Cell counts following protein treatment were also determined for some cell lines.

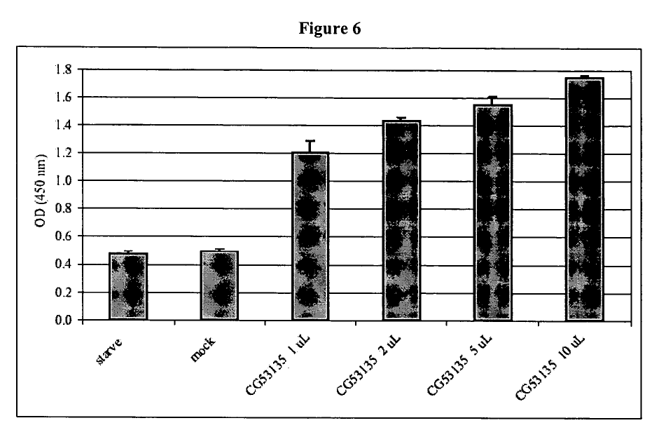

CG53135 was found to induce DNA synthesis in the 786-O human renal carcinoma cell line in a dose-dependent manner (FIG. 6). In addition, CG53135-01 induced DNA synthesis in other cells of epithelial origin, including CCD 1106 KERTr human keratinocytes, Balb MK mouse keratinocytes, and breast epithelial cell line, B5589.

Proliferation of Hematopoietic Cells: no stimulatory effect on DNA synthesis was observed upon treatment of TF-1, an erythroblastic leukemia cell line with CG53135-01. These data suggest that CG53135-01 does not induce proliferation in cells of erythroid origin. In addition, Jurkat, an acute T-lymphoblastic leukemia cell line, did not show any response when treated with CG53135-01, whereas a robust stimulation of BrdU incorporation was observed with serum treatment.

Effects of CG53135 on Endothelial Cells: protein therapeutic agents may inhibit or promote angiogenesis, the process through which endothelial cells differentiate into capillaries. Because CG53135 belongs to the fibroblast growth factor family, some members of which have angiogenic properties, the antiangiogenic or pro-angiogenic effects of CG53135 on endothelial cell lines were evaluated. The following cell lines were chosen because they are cell types used in understanding angiogenesis in cancer: HUVEC (human umbilical vein endothelial cells), BAEC (bovine aortic endothelial cells), HMVEC-d (human endothelial, dermal capillary). These endothelial cell types undergo morphogenic differentiation and are representative of large vessel (HUVEC, BAEC) as well as capillary endothelial cells (HMVEC-d).

CG53135-01 treatment did not alter cell survival or have stimulatory effects on BrdU incorporation in human umbilical vein endothelial cells, human dermal microvascular endothelial cells or bovine aortic endothelial cells. Furthermore, CG53135-01 treatment did not inhibit tube formation, an important event in formation of new blood vessels, in HUVECS. This result suggests that CG53135 does not have anti-angiogenic properties. Finally, CG53135-01 had no effect on VEGF induced cell migration in HUVECs, suggesting that it does no play a role in metastasis.

The above described experiments were also performed using CG53135-02 and CG53135-05 protein products, and the results are summarized in the Conclusion section below.

### Conclusions

Recombinant CG53135-01 induces a proliferative response in mesenchymal and epithelial cells in vitro (i.e., NIH 3T3 mouse fibroblasts, CCD-1070 normal human skin fibroblasts, CCD-1106 human keratinocytes, 786-O human renal carcinoma cells, MG-63 human osteosarcoma cells and human breast epithelial cells), but not in human smooth muscle, erythroid, or endothelial cells. Like CG53135-01, CG53135-02 and CG53135-05 also induce proliferation of mesenchymal and epithelial cells. In addition, CG53135-02 induces proliferation of endothelial cells.

### 6.6 Example 6

### Treatment of Stroke

Thirty male Sprague Dawley rats were allocated to treatment groups as indicated in the study design Table 8 below.

TABLE 8
Experimental Design

| | Number of | | |
|---|---|---|---|
| | Animals | Dose * | Volume * |
| Treatment | Males | (μg) | (μL) |
| Vehicle 1 | 10 | 0 | 50 |
| CG53135-05 E. | 10 | 1 | 50 |
| coli purified | | | |
| product | | | |
| CG53135-05 E. | 10 | 2.5 | 50 |
| coli purified | | | |
| product | | | |

* Administered dose and volume is based on an average bodyweight of 330 g.

### Experimental Procedures

Middle cerebral artery (MCA) Surgery and Intracisternal Injections: Animals were handled for 7 days prior to surgery. Cefazolin sodium (40 mg/kg, i.p) was administered on the day before surgery and just after surgery. At the time of surgery, the rats were anesthetized with 2% halothane in a 2:1 N2O:O2 mixture. Body temperature was maintained at 37±0.50° C. The proximal right MCA was electrocoagulated from just proximal to the olfactory tract to the inferior cerebral vein and was then transected. For intracisternal injections, animals were re-anesthetized as above and placed in a stereotaxic frame. Rats were given CG53135-05 E. coli purified product or vehicle (40 mM acetate, 200 mM mannitol (pH 5.3)) by percutaneous injection into the cisterna magna, once at 1 day, (approximately 24 hours) and once at 3 days, (approximately 72 hours) after MCA. Animals were given test article (2 dose groups) or vehicle treatment according to the study design.

### Clinical Observations/Signs

Animals were observed immediately over a 1 hour period following injections for signs of seizure (indicated by tremor and violent motion about the cage), pain (indicated by loud vocalization), and lethargy. Animals were also observed daily for mortality and moribundity.

Body Weight: Animals were weighed on Days 1, 3, 7, 14 and 21.

Limb Placing Test: limb placing tests were carried out on all animals on Day-1 (pre-operation), Day 1 (just prior to injection), Day 3 and then every 7 days thereafter (Days 7, 14, 21).

Forelimb Placing Test Assessment Score: The forelimb placing test measures sensorimotor function in each forelimb as the animal places the limb on a table top in response to visual, tactile, and proprioceptive stimuli. The forelimb placing test consists of the following evaluations and scoring, where the combined total score for the forelimb placing test reflects a range from 0 (no impairment) to 10 (maximal impairment):

visual placing (forward, sideways): 0-4

tactile placing (dorsal, lateral): 0-4

proprioceptive placing: 0-2

Total score for all forelimb tests: 0-10

Hindlimb Placing Test Assessment Score: Similarly, the hindlimb placing test measures sensorimotor function of the hindlimb as the animal places it on a tabletop in response to tactile and proprioceptive stimuli. The hindlimb placing test consists of the following evaluations and scoring, where the combined total score for the hindlimb placing test reflects a range from 0 (no impairment) to 6 (maximal impairment):

tactile placing (dorsal, lateral): 0-4

proprioceptive placing: 0-2

Total score for all hindlimb tests: 0-6

Body Swing Test: the body swing test was carried out on all animals on Day-1 (pre-operation), Day 1 (just prior to injection), Day 3 and then every 7 days thereafter (Days 7, 14, 21).

The body swing test examines side preference as the animal is held approximately one inch above the surface of the table, and swings to the right or the left side. Thirty swings are counted, and the score is then calculated based on the percentage of swings to the right. (score range=˜50% right swing (no impairment)−0% right swing (maximal impairment))

Cylinder Test: the cylinder test was carried out on all animals on Day-1 (pre-operation) and 7 days thereafter (Days 7, 14, 21). The cylinder test measures spontaneous motor activity of the forelimbs. Animals are placed in a narrow glass cylinder (16.5×25 cm) and videotaped for 5 min on the day before stroke surgery and at weekly intervals thereafter. Videotapes are then scored independently by one experienced observer and up to 50 spontaneous movements will be counted (˜5 min per rat per day). Spontaneous movements include those made by each forelimb to initiate rearing, to land on or to move laterally along the wall of the cylinder, or to land on the floor after rearing.

Macroscopic and Histomorphology: on the day of scheduled termination (Day 3), animals were euthanized by an intraperitoneal injection of Chloral hydrate (500 mg/Kg). Brains were examined grossly and removed, postfixed in formalin, dehydrated and embedded in paraffin. Coronal sections (5 mm) will be cut on a microtome mounted on to glass slides, and stained with hematoxylin/eosin (H&E). The area of cerebral infarcts on each of seven slices (+4.7, +2.7, +0.7, −1.3, −3.3, −5.3, and −7.3 mm compared with Bregma) was determined using a computer interface imaging system using the indirect method (area of the intact contralateral hemisphere—area of the intact ipsilateral hemisphere) to correct for brain shrinkage during processing. Infarct volume was then expressed as a percentage of the intact contralateral hemispheric volume. Volumes of the infarction in the cortex and striatum were also determined separately using these same methods. H&E stained section was examined for histological changes such as hemorrhage, abscess or tumor formation.

Statistical Analysis: all intracisternal injections, behavioral testing, and subsequent histological analyses were done by investigators blinded to the treatment assignment of each animal. Data are then expressed as means +/−SEM, and will be analyzed by one or two way (ANOVA) followed by appropriate pairwise post hoc tests with correction for multiple comparisons.

### Results

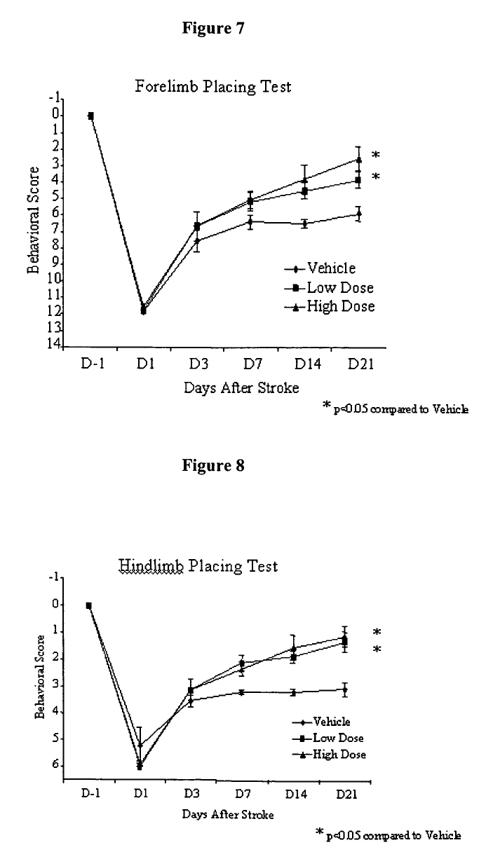

Forelimb Placing Test: on days-1, 1, 3, 7, 14, and 21 relative to MCA occlusion, animals were examined by using a limb placing test to assess sensorimotor function in the forelimb in response to visual, tactile and proprioceptive stimuli (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60.) Visual placing (scored 0-4), tactile placing (scored 0-4), and proprioceptive placing (scored 0-2) were summed to generate a range of potential total scores from 0 to 12, with 12 representing maximal impairment (FIG. 7).

Hindlimb Placing: on days-1, 1, 3, 7, 14, and 21 relative to MCA occlusion, animals were examined by using a limb placing test to assess sensorimotor function in the hindlimb in response to tactile and proprioceptive stimuli (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60). Tactile placing (scored 0-4), and proprioceptive placing (scored 0-2) were summed to generate a range of potential total scores from 0 to 6, with 6 representing maximal impairment (FIG. 8).

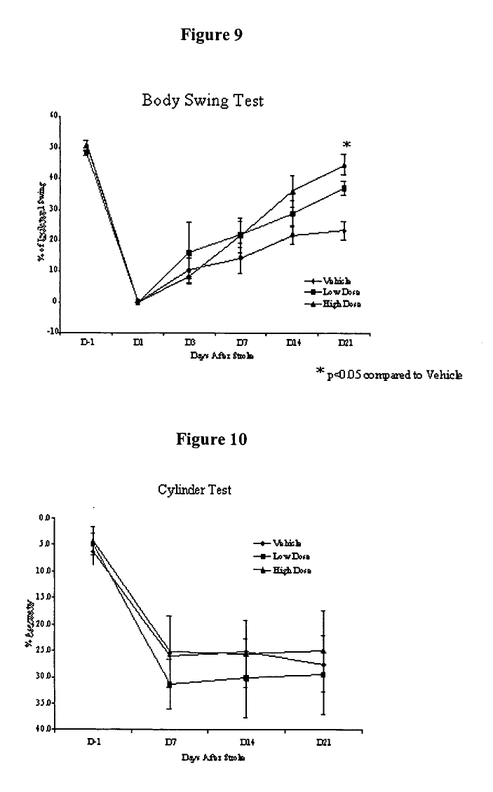

Body Swing Test: On days-1, 1, 3, 7, 14, and 21 relative to MCA occlusion, animals were examined by using a body swing test to assess side preference as the animal is held approximately one inch above the surface of the table, and swings to the right or the left side. (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60.) Thirty swings were counted, and the score calculated based on the percentage of swings to the right (FIG. 9).

Cylinder Test: On days-1, 1, 3, 7, 14, and 21 relative to MCA occlusion, animals were examined by cylinder test to assess spontaneous motor activity of the forelimbs (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60.) Briefly, animals are placed in a narrow glass cylinder (16.5×25 cm) and videotaped for 5 min on the day before stroke surgery and at weekly intervals thereafter. Videotapes are then scored independently by one experienced observer and up to 50 spontaneous movements will be counted (˜5 min per rat per day). Spontaneous movements include those made by each forelimb to initiate rearing, to land on or to move laterally along the wall of the cylinder, or to land on the floor after rearing (FIG. 10).

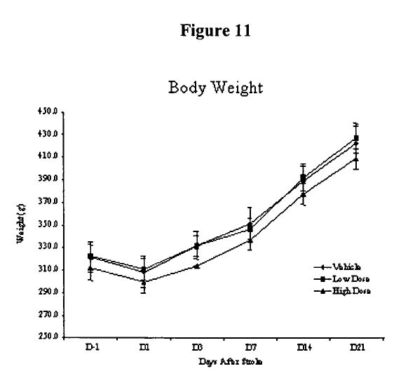

Body Weight: animals were weighed on days-1, 1, 3, 7, 14, and 21 relative to MCA occlusion and the results indicate no significant difference between the vehicle and CG53135-05 treatment (FIG. 11).

### Conclusion

Administering CG53135 following MCA occlusion suggested that both the low and high doses produced a significant enhancement of recovery on forelimb (FIG. 7) and hindlimb placing tests (FIG. 8) for the contralateral (affected) limbs, and improvement on the body swing test (FIG. 9). This pattern of activity with other therapeutics in this model has generally been shown to reflect improvement in cerebrocortical and subcortical (striatal) function, respectively (Dijkhuizen R M, Ren J, Mandeville J B, Wu O, Ozdag F M, Moskowitz M A, Rosen B R, Finklestein S P. 2001, Proc Natl Acad Sci USA 98(22):12766-71). No apparent differences were seen on the cylinder test (FIG. 10) of spontaneous limb use or on animal body weight (FIG. 11).

Therefore, CG53135 administration will be useful in the treatment of pathological conditions including ischemic stroke, hemorrhagic stroke, trauma, spinal cord damage, heavy metal or toxin poisoning and neurodegenerative diseases (such as Alzheimer's, Parkinson's Disease, Amyotrophic Lateral Sclerosis, Huntington's Disease).

### 6.7 Example 7

### Matrix Metalloproteinase Production Assay

The matrix metalloproteinases (MMPs) are a family of related enzymes that degrade the extracellular matrix in bone and cartilage. These enzymes operate during normal development in tissues differentiation and remodeling. In arthritic diseases, such as Osteoarthritis (OA) and Rheumatoid Arthritis (RA), elevated expression of these enzymes contributes to irreversible matrix degradation. Thus, effect of CG53135 on MMP production was assayed.

The activity of CG53135 on matrix metalloproteinase (MMP) production was assessed using the SW1353 chondrosarcoma cell line (ATCC HTB-94). This cell line is a well-established chondrocytic cellular model for matrix metalloproteinases (MMP) production. SW1353 cells were plated in a 24-well plate at 1×105 cells/ml (1 ml) in DMEM medium-10% FBS. Following overnight incubation, the medium was replaced with DMEM+0.2% Lactabulmin serum. CG53135-05 E. coli purified product was added to the wells at doses ranging from 10 to 5000 ng/ml, in the absence or presence of IL-1 beta (0.1 to 1 ng/ml, R&D systems Minneapolis, Minn.), TNF-alpha (10 ng/ml, R&D systems) or vehicle control to a final volume of 0.5 ml. IL-1 beta and TNF-alpha are both potent stimulators of MMP activity. All treatments were done in triplicate wells. After 24 hours, the supernatants were collected and Pro-MMP-1, and -13, as well as TIMP-1 (tissue inhibitor of matrix metalloproteinase), a natural inhibitor of MMP activity, was measured by ELISA (R&D systems). The measurements were normalized to the number of cells by an MTS assay.

### Results

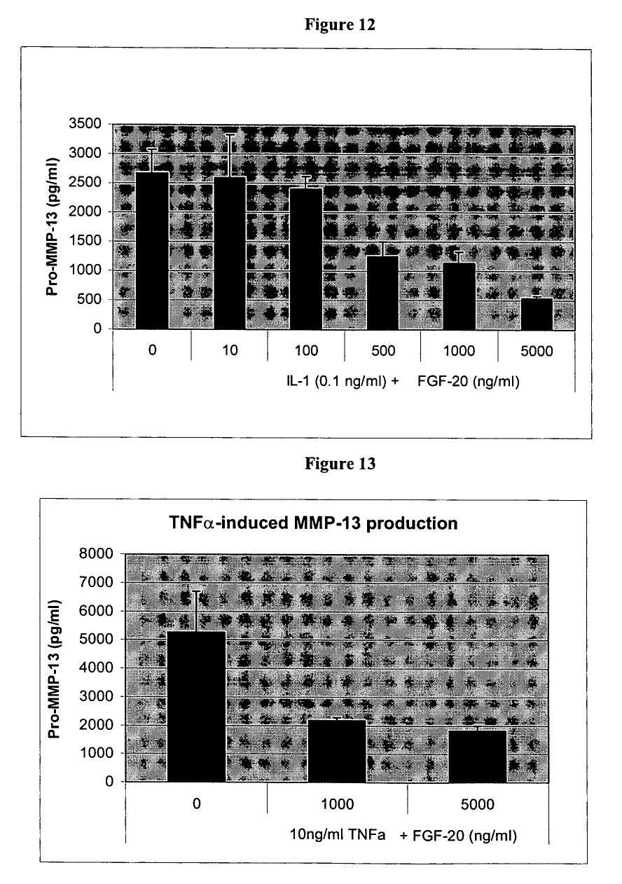

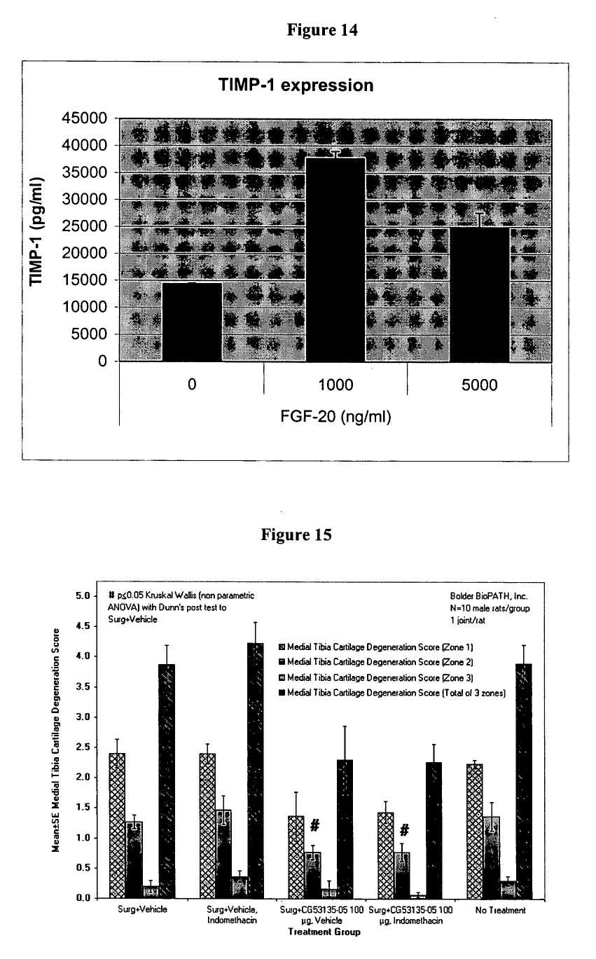

CG53135 significantly decreased MMP-13 production in the presence of either IL-1 beta or TNF-alpha as demonstrated in FIG. 12 and FIG. 13, respectively. IL-1 beta and TNF-alpha are both potent stimulators of MMP activity. MMP-13 affinity for type II collagen, the main collagen that is degraded in OA, is ten times higher that of MMP-1. Since MMP-13 expression increases in OA and RA, the decrease of MMP-13 observed with addition of CG53135 indicates that the protein can be used as an OA and RA therapeutic. Furthermore, CG53135 up-regulated the production of TIMP-1, a natural inhibitor of MMP activity (FIG. 14). This enhancement of TIMP-1 production by CG53135 is beneficial in reducing the matrix breakdown by MMP-1 and -13 observed in OA and RA. In addition, CG53135 had no effect on MMP-3 production constitutively or after IL-1 induction. Similarly, CG53135-05 E. coli purified product showed increase in basal expression of MMP-1 in SW1353 cells.

### 6.8 Example 8

### Effect of CG53135 on Normal Rats: Proof of Principle to the Meniscal Tear Model

The effect of CG53135 on the normal rats was studied as a proof of principle to drive further studies in disease model (ex: meniscal tear model of osteoarthritis in rats). The effect of CG53135 on synovium and cartilage was assessed by injecting the protein into normal male Lewis rats.

Effects of Intra-Articular Injection of CG53135-05 E. coli Purified Product in Normal Rats

The rats were injected intra-articularly three times per week for 2 weeks with vehicle solution (8 mM acetate, 40 mM arginine, and 0.6% glycerol (pH 5.3) in approximately 1% hyaluronic acid), 10 μg CG 53135-05 E. coli purified product or 100 μg CG 53135-05 E. coli purified product.

Study Design: Male Lewis rats weighing 293-325 grams on day 0 were obtained from Harlan Sprague Dawley (Indianapolis, Ind.) and acclimated for 8 days. The rats were divided into three treatment groups with three animals in each group: two groups received CG53135 and one received only the vehicle control. The rats were anesthetized with isoflurane and injected through the patellar tendon into the area of the cruciate attachments of both knees. CG53135 was injected at doses of 0.1 mg/ml (0.01 mg/joint) or 1.0 mg/ml (0.1 mg/joint). Controls were injected with the vehicle solution as described above. Injections were done Monday, Wednesday and Friday for 2 weeks. The animals were terminated on day 15 at which time they were injected ip with BRDU (100 mg/kg) in order to pulse label proliferating cells.

### Observations and Analysis of Markers of Pathology

Gross observations: Rats were observed daily for abnormal swelling or gait alterations and were weighed weekly.

Histopathology: Preserved and decalcified (5% formic acid) knees were trimmed into 2 approximately equal longitudinal (ankles) or frontal (knees) halves, processed through graded alcohols and a clearing agent, infiltrated and embedded in paraffin, sectioned, and stained with toluidine blue (knees). Multiple sections (3 levels) of right knee were analyzed microscopically with attention to the parameters of interest listed below. Each parameter was graded as normal, minimal, mild, moderate, marked or severe. Evaluation of the cartilage was done using descriptive parameters rather than the scoring criteria generally used in the osteoarthritis model because of the type of alterations generated by the repetitive injection of the protein. Although animals were injected with BRDU prior to termination, the proliferative changes were readily observed in toluidine blue stained sections.

### Results

TABLE 9
Microscopically Monitored Parameters

| | | Central Cruciate | |
|---|---|---|---|
| Synovial | Cartilage | Attachment Area | Chondro- |
| Alterations | Alterations | Alterations | genesis |
| hyperplasia | cartilage | inflammation and | marginal |
| infiltration | proteoglycan | fibroplasia | zone or |
| of synovium | loss | bone or cartilage | periosteal |
| with macrophages | cartilage | damage | chondro- |
| fibroplasia | fibrillation | | genesis |
| matrix | | | |
| (proteoglycan | | | |
| deposition | | | |
| in fibrotic | | | |
| synovium) | | | |

Live Phase Parameters Body weights were similar in vehicle and protein injected animals throughout the study (Table 9). Knees injected with 100 μg of protein had some evidence of fibrosis clinically during the injection process beginning with the 3rd injection.

Morphologic Pathology: Vehicle injected rats had minimal to mild synovial hyperplasia, inflammation and fibroplasia with none to minimal matrix deposition in fibrotic synovium. Articular cartilage had no proteoglycan loss or fibrillation. The central area of the joint where the cruciates attach and in which the intra-articular injections are made had none to minimal fibroplasia and cartilage/bone damage. No marginal zone chondrogenesis was present.

Knees injected with 100 μg CG 53135-05 E. coli purified product had mild to moderate synovial hyperplasia, inflammation and fibroplasia with minimal to moderate matrix deposition in fibrotic synovium. Articular cartilage had no proteoglycan loss or fibrillation. The central area of the joint where the cruciates attach and in which the intra-articular injections are made had none to minimal fibroplasia and cartilage/bone damage. One knee had minimal marginal zone chondrogenesis.

Knees injected with 100 μg CG 53135-05 E. coli purified product had moderate to marked synovial hyperplasia, inflammation and fibroplasia with moderate matrix deposition in fibrotic synovium. Articular cartilage had none to minimal proteoglycan loss or fibrillation. The central area of the joint where the cruciates attach and in which the intra-articular injections are made had minimal to marked fibroplasia and cartilage/bone damage. All knees had mild to moderate marginal zone chondrogenesis. One animal had chondrogenesis in areas associated with articular cartilage.

### Conclusion

These results demonstrate that repetitive intra-articular injection of CG53135 induces synovial fibroplasia and chondrogenesis. Vehicle injections resulted in mild inflammation and fibroplasia thus suggesting that this vehicle has some irritant potential. Concentration responsive increases in synovial proliferative response as well as marginal zone chondrogenesis occurred in animals injected with protein. The area of the cruciate attachment where injections occurred had areas of bone resorption and fibroplasia which also increased in severity with increasing concentrations of the protein as did the synovial inflammation. The potentially adverse effects of observed synovial fibroplasia and bone resorption could have been due to either FGF-20 activity or endotoxin levels within the non-clinical grade hyaluronic acid used to formulate the protein. In addition, inflammation in the joint can induce bone resorption and marginal zone chondrogenesis so these results need to be interpreted in light of the possibility that the inflammatory response to the protein injection contributed to the proliferative response. The morphologic appearance of the proliferative changes and chondrogenesis clearly indicates that the biological activity of this protein (CG53135) is important in generating the response.

The results of the experiments reported herein indicate repetitive intra-articular injection of CG53135 induces synovial fibroplasia and chondrogenesis.

### 6.9 Example 9

### Intra-Articular Injection of CG53135-05 in Meniscal Tear Model of Rat Osteoarthritis: Prophylactic and Therapeutic Dosing

Example 8 utilized CG53135 administration into the joints of normal rats to identify effects on relevant cell populations by histomorphometric analysis. At the dose of 100 ug/joint, CG53135 induced significant marginal zone chondrogenesis similar to that seen with other growth factors such as TGF-beta, suggesting an effect on pluripotent stem cells within the marginal zone. There was no apparent effect on mature chondrocytes as evidenced by the lack of a response in the mature cartilage areas of the joints. The potentially adverse effects of observed synovial fibroplasia and bone resorption could have been due to either FGF-20 activity or endotoxin levels within the non-clinical grade hyaluronic acid used to formulate the protein.

Further studies in osteoarthritic animals performe addressed the following: (1) synergy with an anti-inflammatory drug (standard approach for osteoarthritis patients); (2) whether CG53135 can induce functional repair or protection of joint cartilage layers; and (3) whether synovial fibroplasia and bone resorption were CG53135-induced or due to contaminating endotoxin within the formulation.

Thus one aspect of this study was to evaluate the protective and therapeutic effects of intra-articular injection of CG53135 on joint damage in osteoarthritis in the meniscal tear model of rat osteoarthritis. This relatively new model of OA has been shown to have morphologic alterations of cartilage degeneration and osteophyte formation that resemble changes occurring in spontaneous disease and surgically induced disease in other species (Bendele, A. M., Animal Models of Osteoarthritis. J. Musculoskel. Neuron Interact. 2001; 1:363-376, Bendele, A. M. and Hulman, J. F. Spontaneous cartilage degeneration in guinea pigs. Arthritis Rheum. 1988; 31:561-565). The model can be used to evaluate potential beneficial effects of anti-degenerative as well as regenerative therapies.

### Experimental Design

Animals (10/group), housed 2/cage, were anesthetized with isoflurane and the right knee area is prepared for surgery. A skin incision was made over the medial aspect of the knee and the medial collateral ligament was exposed by blunt dissection, and then transected. The medial meniscus was then reflected medially with a fine scissor and a cut was made through the full thickness to simulate a complete tear. The skin was closed with suture.

Prophylactic Dosing: intra-articular dosing (CG53135-05 E. coli purified product) of the right knee joint was initiated on the day of surgery and is continued for 2 weeks post-surgery with intra-articular injections given Thursday, Saturday, and Monday (day 0, 2, 4, 7, 9, and 11) with rats under Isoflurane anesthesia. Indomethacin, a nonsteroidal anti-inflammatory drug, was dosed (1 mg/kg/day) daily by the oral route starting on the day of surgery to reduce any potential inflammation due to the injection. Body weights were recorded on days 0, 7 and 14. After animal termination on day 14 post-surgery, both knees were collected for histopathologic evaluation. The study design is shown in Table 10.

TABLE 10
Prophylactic Dosing Study Design

| | CG53135-05 | Co-therapy | Number of Animals |
|---|---|---|---|
| Group | Treatment^{a} | Treatment^{b} | Males |
| 1 | Vehicle | Vehicle | 10 |
| | (intra-articular) | | |
| 2 | Vehicle | Indomethacin | 10 |
| | (intra-articular) | | |
| 3 | CG53135-05 E. coli | Vehicle | 10 |
| | purified product | | |
| | (intra-articular) | | |
| 4 | CG53135-05 E. coli | Indomethacin | 10 |
| | purified product | | |
| | (intra-articular) | | |
| 5 | None | None | 10 |

^{a}Administration 3 times per week for 2 weeks (100 μg/joint, intra-articular)
^{b}Administration daily for 2 weeks (0.5 mg/kg, PO)

Therapeutic Dosing: intra-articular dosing (CG53135-05 E. coli purified product) of the right knee joint is initiated on day 21 of post-surgery and is continued for 2 weeks with intra-articular injections given Friday, Sunday, and Tuesday (day 22, 25, 27, 29, 32, and 34) with rats under isoflurane anesthesia. Indomethacin is dosed daily by the oral route starting on the day of surgery. Body weights are recorded on days 0, 7, 14, 21, 28, and 35. On day 35, both knees are collected for histopathologic evaluation. The study design is shown in Table 11.

TABLE 11
Therapeutic Dosing Study Design

| | CG53135-05 E. coli | | Number of |
|---|---|---|---|
| | purified product | Co-therapy | Animals |
| Group | Treatment^{a} | Treatment^{b} | Males |
| 1 | Vehicle | Vehicle | 10 |
| | (intra-articular) | | |
| 2 | Vehicle | Indomethacin | 10 |
| | (intra-articular) | | |
| 3 | CG53135-05 E. coli | Vehicle | 10 |
| | purified product | | |
| | (intra-articular) | | |
| 4 | CG53135-05 E. coli | Indomethacin | 10 |
| | purified product | | |
| | (intra-articular) | | |
| 5 | None | None | 10 |

^{a}Administration 3 times per week for 2 weeks (100 μg/joint, intra-articular)
^{b}Administration daily for 2 weeks (0.5 mg/kg, PO)

Results of prophylactic dosing study: Observations made include the standards followed for this model. Multiple sections (3 levels) of right knee were analyzed microscopically and scored according to the following methods. In scoring the 3 sections, the worst case scenario for the 2 halves on each of the 3 slides representing 3 levels was determined for cartilage degeneration and osteophyte formation. This value for each parameter for each slide was then averaged to determine overall subjective cartilage degeneration scores for tibia and femur and osteophyte scores for tibia.

Cartilage degeneration was scored none to severe (numerical values 0-5) for depth and area (surface divided into thirds) using the following criteria:

0=no degeneration
1=minimal degeneration, chondrocyte and proteoglycan loss with or without fibrillation involving the superficial zone
2=mild degeneration, chondrocyte and proteoglycan loss with or without fibrillation involving the upper ⅓
3=moderate degeneration, chondrocyte and proteoglycan loss with fibrillation extending well into the midzone and generally affecting ½ of the total cartilage thickness
4=marked degeneration, chondrocyte and proteoglycan loss with fibrillation extending well into the deep zone but without complete (to the tidemark) loss of matrix
5=severe degeneration, matrix loss to tidemark

Strict attention to zones (outside, middle, inside thirds) was adhered to in this scoring method and the summed scores reflect a global summation of severity of tibial degeneration.

In addition to this overall subjective analysis of cartilage degeneration, an additional subjective assessment was done using similar criteria to evaluate severity of degeneration but with attention to specific regional differences across the tibial plateau. In this OA model, generally the outside ⅓ of the tibia is most severely affected by the meniscal tear injury with lesions often extending to the tidemark by 3 weeks post-surgery. The middle ⅓ is usually a transition zone where severe or marked change becomes moderate or mild and the inner ⅓ seldom has changes greater than mild or minimal. In an attempt to determine potential differences of treatment on the severe lesion of the outside ⅓ vs. the milder lesions of the middle ⅓ and inside ⅓, these regions were each scored separately. The sum of the regional values was calculated and expressed as sum of 3 zones.

In addition to the above subjective scoring, a micrometer measurement of total extent of tibial plateau affected by any severity of degeneration (Total Tibial Cartilage Degeneration Width μm) extended from the origination of the osteophyte or marginal zone if no osteophyte was present with adjacent cartilage degeneration (outside ⅓) across the surface to the point where tangential layer and underlying cartilage appeared histologically normal.

An additional measurement (Significant Cartilage Degeneration Width μm) reflected areas of tibial cartilage degeneration in which chondrocyte and matrix loss extended through greater than 50% of the cartilage thickness.

Finally, a micrometer depth of any type of lesion (cell/proteoglycan loss, change in metachromasia, but may have good retention of collagenous matrix and no fibrillation) expressed as a ratio of depth of changed area vs. depth to tidemark was included and taken over 4 equally spaced points on the tibial surface. These measurements were taken (1st) matrix adjacent to osteophyte (2nd) ¼ of the distance across the tibial plateau (3rd) ½ of the distance across the tibial plateau (4th) ¾ of the distance across the tibial plateau. This measurement was the most critical analysis of any type of microscopic change present. The depth to tidemark measurement (denominator) also gives an indication of cartilage thickness across the tibial plateau and therefore allows comparisons across groups when trying to determine if hypertrophy or hyperplasia has occurred.

A single tibial growth plate measurement was taken for each section in an area thought to best represent the overall width in the non tangential plane of the section.

Scoring of the osteophytes and categorization into small, medium and large was done with an ocular micrometer.

None=0 no measurable proliferative response at marginal zone
Small osteophytes=1 (up to 299 μm)
Medium osteophytes=2 (300-399 μm)
Large osteophytes=3 (>400 μm)

The score (0-3) was included in the overall joint score. In addition, the mean±SE for the actual osteophyte measurement (average for 3 sections) was also determined.

Generally, in doing the surgery, attempts were made to transect the collateral ligament at a location that results in the meniscus being reflected proximally toward the femur. The cut was then made by inserting the scissors tip toward the femur rather than the tibia. Some mechanical damage may then be detected in the femoral condylar cartilage but is rarely encountered on the tibia, thus making the tibia the most appropriate site for assessment of chondroprotection.

Focal small areas of proteoglycan and cell loss that were likely a result of physical trauma to the femoral cartilage were described but not included in the score with larger more diffuse areas receiving subjective scores according to methods described for the tibia. These larger areas were more consistent with non traumatic degeneration. Because of the possibility of iatrogenic lesions on the femur, overall joint scores were expressed both with and without femoral cartilage degeneration scores.

Damage to the calcified cartilage layer and subchondral bone was scored using the following criteria:

0=No changes
1=Increased basophilia at tidemark: no fragmentation of tidemark or marrow changes
2=Increased basophilia at tidemark: minimal to mild fragmentation of calcified cartilage of tidemark, mesenchymal change in marrow involves ¼ of total area but generally is restricted to subchondral region under lesion
3=Increased basophilia at tidemark: Mild to marked fragmentation of calcified cartilage, Mesenchymal change in marrow is up to ¾ of total area, Areas of marrow chondrogenesis may be evident but no collapse of articular cartilage into epiphyseal bone
4=Increased basophilia at tidemark: Marked to severe fragmentation of calcified cartilage, Marrow mesenchymal change involves up to ¾ of area and articular cartilage has collapsed into the epiphysis to a depth of 250 μm or less from tidemark
5=Increased basophilia at tidemark: Marked to severe fragmentation of calcified cartilage, Marrow mesenchymal change involves up to ¾ of area and Articular cartilage has collapsed into the epiphysis to a depth of greater than 250 μm from tidemark

Descriptive comments were made on degree of synovial inflammation, synovial fibrosis, marginal zone chondrogenesis, bone resorption, fibrous overgrowth with or without chondrogenesis/incorcoration into existing cartilage

Statistical Analysis: statistical analysis of histopathologic parameters was done by comparing group means using the Student's two-tailed t-test with significance set at p≦0.05. Because of the nature of the data, a non Parametric ANOVA (Kruskal-Wallis test) was used to analyze the scored parameters and a parametric ANOVA was used to analyze the measurements. The appropriate post test used was Dunnett's multiple comparisons test on the parametric data and a Dunn's test was used on the non parametric data. Significance was set at p≦0.05 for all parameters.

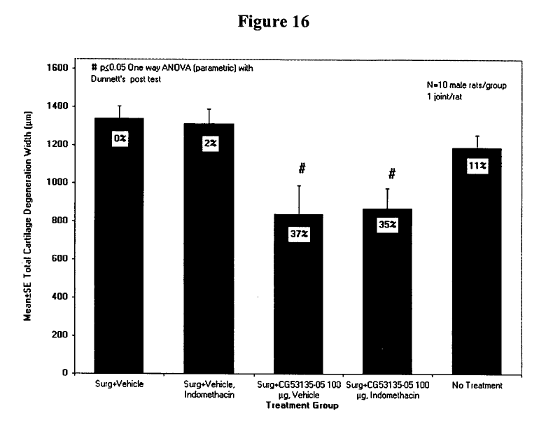

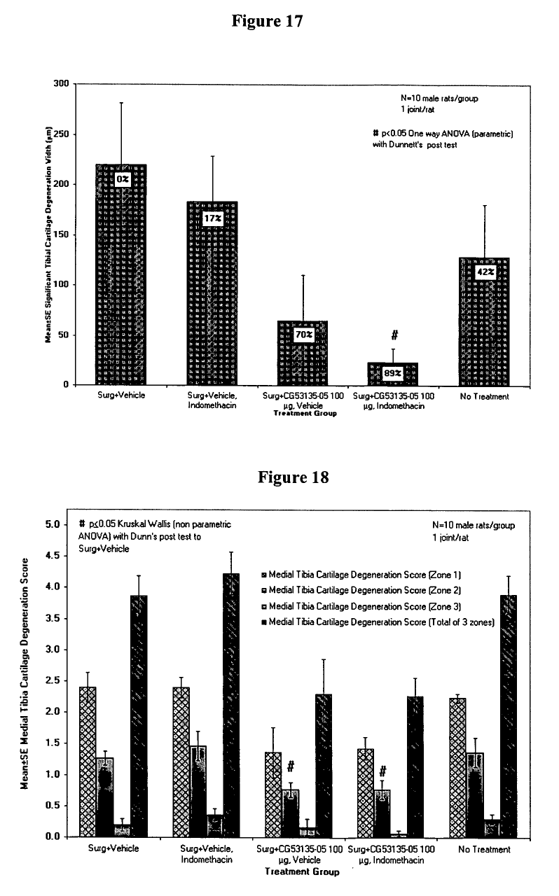

Results: intra-articular injection of 100 μg CG53135-05 E. coli purified product with or without concurrent indomethacin administration resulted in significant inhibition (39%) of tibial cartilage degeneration on the middle ⅓ (40-43% for zone 1) and an overall insignificant inhibition of the summed 3 zones of 41% (FIG. 15). Total cartilage degeneration width was significantly decreased 35-37% (FIG. 16) and significant degeneration was reduced 70-89% with this inhibition being significant only in the group treated with protein and indomethacin (FIG. 17).

Results of the prophylactic dosing study: the data described indicate that intra-articular injection of 100 μg of CG53135-05 E. coli purified product in knee joints of rats with medial meniscal tear results in chondroprotective effects as a result of both inhibition of cartilage degeneration and stimulation of cartilage repair. Some joints had layering of proliferated new cartilage over existing normal appearing or damaged cartilage. This observation is particularly exciting as it demonstrates the potential for resurfacing to occur.

These beneficial effects were always associated with diffuse synovial fibroplasia, bone resorption and increased synovial inflammation. Concurrent indomethacin treatment (1 mg/kg/day) had minimal if any effect on the disease process in knees injected with Synvisc alone or the disease process and reaction to the protein in knees injected with Synvisc containing protein. The single exception to this statement is reflected in the data for osteophyte measurements where all groups had similar measurements except the group treated with protein and vehicle po. This group had greater measurements thus suggesting greater marginal zone stimulation, not an uncommon occurrence in inflamed joints.

The morphologic changes induced by injection of 100 μg of this protein demonstrate the potential for CG53135 to be effective in cartilage repair processes. It has the capacity to induce fibrous tissue proliferation with differentiation to cartilage and importantly, integration of that newly proliferated tissue. The proliferative processes are somewhat disorganized and counter productive in areas such as the marginal zone and subchondral bone. However, rodents definitely have much greater propensity to exhibit marginal zone, periosteal and marrow proliferation from a variety of stimuli including inflammatory mediators so some of the excessive and counter productive responses seen in rats might not occur in dogs or primates. Also, there may have been some induction of an antibody response thus leading to enhanced knee inflammation that would not occur in humans or other animals that did not have an antibody response.

Additional studies that are useful in delineating the potential efficacy of CG53135 in osteoarthritis include: (1) evaluation in an animal model, e.g., a rabbit or a dog model of OA-this would allow evaluation in a larger joint with cartilage and bone structure that is more similar to humans and species that has less tendency to exhibit hyperproliferative responses such as those that occur in rodents; and (2) evaluation of ia injections for 3-4 weeks, possibly with more aggressive anti-inflammatory systemic therapy followed by a recovery period to see how the new tissue remodels would be interesting. It may be that allowing the joint to remodel with no further proliferative stimulus would result in a more pleasing morphologic endpoint. Alternatively, the time point of which the immune response that would clear may result in a endpoint. Cycles of treatment with periods of remodeling might be the way to achieve the most satisfactory repair. Studies such as these would also answer the question of whether the repair tissue will hold up long term. Generally fibrocartilage has less of a tendency to do this.

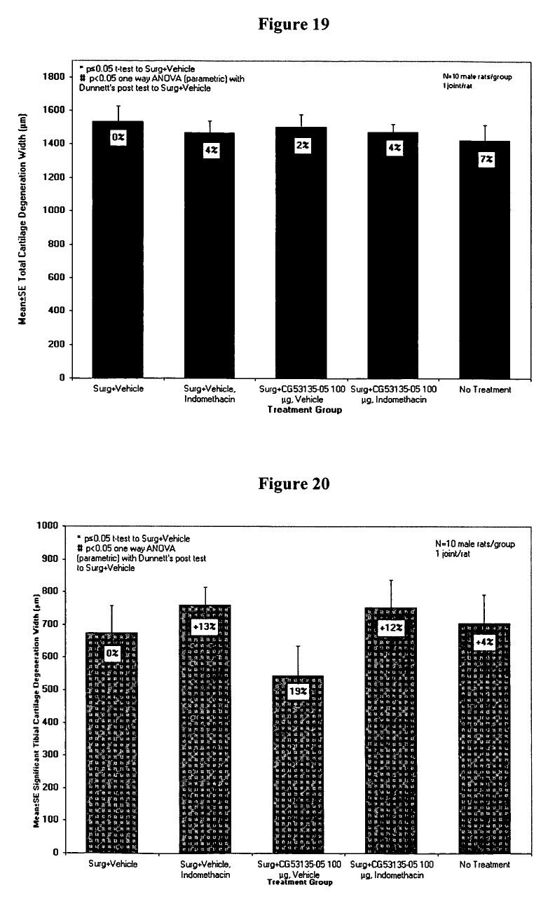

Results: Intra-articular injection of 100 μg CG53135-05 E. coli purified product with or without concurrent oral indomethacin administration did not result in significant inhibition of tibial cartilage degeneration scores (FIG. 18). Total or significant cartilage degeneration width was not decreased (FIGS. 19, 20).

Results of the therapeutic dosing study: The data described demonstrated the potential chondroproliferative activities of CG53135 administered intra-articularly. However, protein injected joints had markedly increased inflammation, fibroplasia and connective tissue resorptive process.

The most important difference between the prophylactic and therapeutic dosing studies was the nature of the OA lesion at the time of initiation of dosing. Rats in the therapeutic dosing study had an area of severe matrix loss in the outer to middle ⅓ of the cartilage thus exposing the calcified cartilage/subchondral bone to the protein. Effective repair thus required filling of this defect with newly proliferated tissue coming from the marginal zone or exposed marrow pleuripotential cells. In the prophylactic dosing study, beneficial effects required inhibition of matrix degradation and stimulation of repair on a degenerating scaffold with repair tissue originating from the marginal zone only. Since the filling of a defect would be much more difficult than repairing a damaged scaffold, it may be that a longer duration of treatment would be required in a therapeutic model in order to see beneficial effects.

Indomethacin treatment was not effective in reducing the inflammatory changes and it had no beneficial effects on inhibiting the resorptive processes occurring in bone. In order to achieve effective proliferation and differentiation to cartilage in the absence of inflammation and tissue destruction, following modification to the therapeutic dosing study can be attempted: Increasing the dosing interval to once or twice weekly and/or increasing the study duration to allow time for the proliferative tissue to fill the large cartilage defects induced by this disease process. Another possibility is to investigate the effects of CG53135 in a larger species such as the dog as dogs have less of a tendency to proliferate connective tissue and resorb bone in response to various stimuli than rodents.

The results detailed herein (both prophylactic and therapeutic dosing studies) indicate that CG53135 has specific utility in severely osteoarthritic joints that are destined for joint replacement. These types of agents would be injected into joints that have little or no normal cartilage remaining and are in need of resurfacing. In this situation, repair could originate from pleuripotential cells in the marginal zones or bone marrow. Repair originating from these locations will likely result in production of fibrocartilage rather than hyaline cartilage. However, some cartilage would be preferable to no cartilage and it may be that an injectable method of sustaining a cartilage surface would be acceptable even though treatments would likely have to be repeated over time to sustain the repair. Treatments with injectable anabolic agents will likely require some kind of cyclical process in conjunction with continuous passive motion rather than sustained active load bearing motion.

### 6.10 Example 10

### CG53135 Rescues Neuronal PC12 Cells from Serum-Starvation Induced Cell Death

To assess the trophic (neuroprotective) qualities of CG53135 and compare to the action of Nerve Growth Factor (NGF) and Epidermal Growth factor (EGF), the following experiment was performed.

### Materials and Method:

Materials: PC12 cells, tissue culture plates and medium (DMEM+/−10% FBS), NGF, EGF, CG53135-05 E. coli purified product (by Process 1).

Method: Plate PC12 cells at low density on poly-lysine coated tissue culture plates in DMEM+10% FBS. Culture 24 hours. Administer serum-free media containing NGF or CG53135 at a range of doses or no growth factor supplements. Photograph at 72 hours to visualize cell survival and proliferation.

### Results:

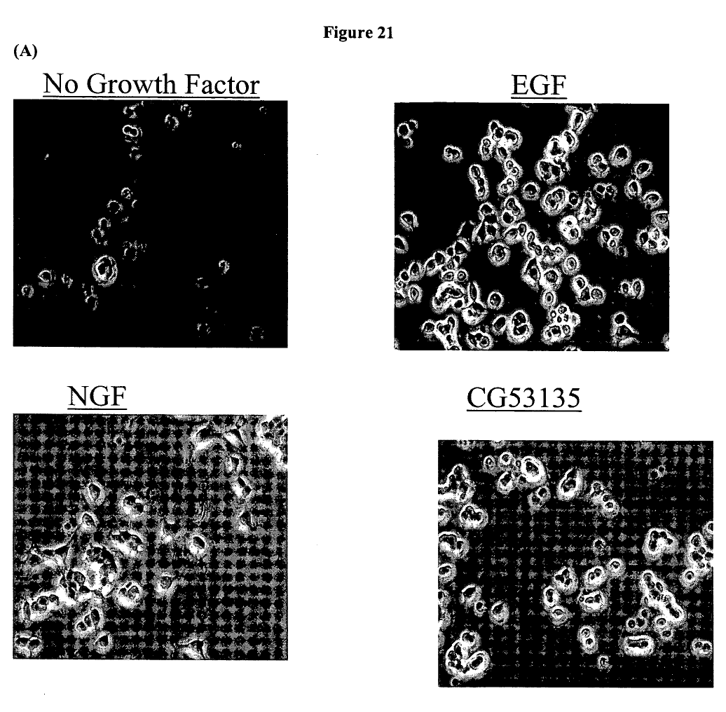

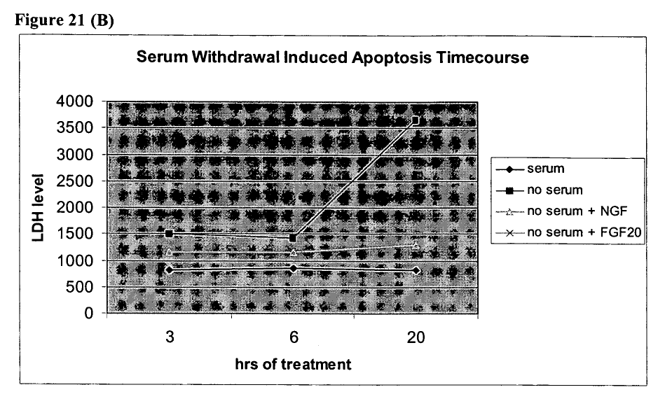

CG53135 prevented cell death in a dose-dependent fashion. The maximal trophic activity was achieved at 50 ng/ml. The potency of CG531345 was approximately 20% of the potency of NGF, however the maximal extent of trophic action by both growth factors was equivalent. EGF also exhibited trophic activity. Cell death (apoptosis) was measured by LDH assay and visually. (FIG. 21)

### Conclusion:

CG53135 acts similarly to the neurotrophin NGF and to the growth factor EGF to the extent that CG53135 is capable of rescuing PC12 cells from serum starvation-induced cell death. Thus, CG53135 possesses trophic activity. Trophic activity is recognized to have value in the treatment of numerous disorders of the central nervous system. In particular, the ability to protect neuronal cells is important to diseases where neurodegeneration is involved, such as Alzheimer's disease, Parkinson's disease and diseases with catastrophic cell death such as stroke and traumatic brain injury.

### 6.11 Example 11

### CG53135 Inhibits Serum-Withdrawal Induced Caspase Activation in Neuronal PC12 Cells

This experiment was performed to assess the ability of CG53135 to inhibit the activation of pro-apoptotic caspace enzymes, and compare to the action of Nerve Growth Factor (NGF).

### Materials and Method:

Materials: PC12 cells, tissue culture plates and medium (DMEM+/−10% FBS), NGF, CG53135-05 E. coli purified product (by Process 1).

Method: Plate PC12 cells at medium density on poly-lysine coated tissue culture plates in DMEM+10% FBS. Culture 24 hrs. Administer serum-free media containing NGF (100 ng/ml) or CG53135 (1000 ng/ml) or no growth factor supplements. Collect cell lysates at various time points (0, 3, 6 and 20 hrs.). Evaluate caspase 2, 3, 8, 9 activation by ELISA.

### Results:

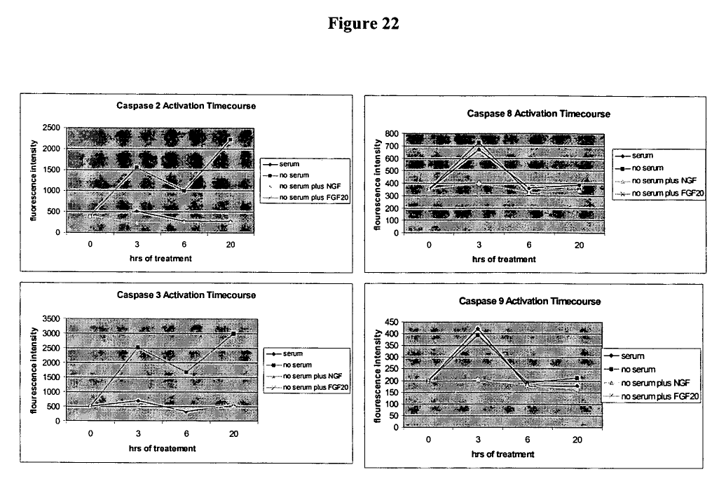

Serum withdrawal induced caspase activity time-dependently. Both CG53135 and NGF blocked caspase induction. (FIG. 22)

### Conclusion:

CG53135 acts similarly to the neurotrophin NGF to the extent that both proteins are able to prevent the activation of apoptosis promoting caspase enzymes upon apoptotic stimuli. Caspases have been implicated in a number of diseases of the CNS involving neuronal death, including Alzheimer's disease, Parkinson's disease, stroke and traumatic brain injury. Therefore, CG53135 has value in the treatment of these diseases.

### 6.12 Example 12

### CG53135 Induces Neurite Outgrowth by Neuronal PC12 Cells

This experiment was performed to assess the neuritogenic qualities of CG53135-05 and compare to the action of Nerve Growth Factor (NGF)

### Materials and Method:

Materials: PC12 cells, tissue culture plates and medium (DMEM+/−10% FBS), NGF, CG53135-05 E. coli purified product (by Process 1).

Method: Plate PC12 cells at low density on poly-lysine coated tissue culture plates in DMEM+10% FBS. Culture 24 hrs. Administer serum-free media containing NGF or CG53135 at 100 ng/ml or no growth factor supplements. Photograph at 72 hrs to visualize neurite outgrowth.

### Results:

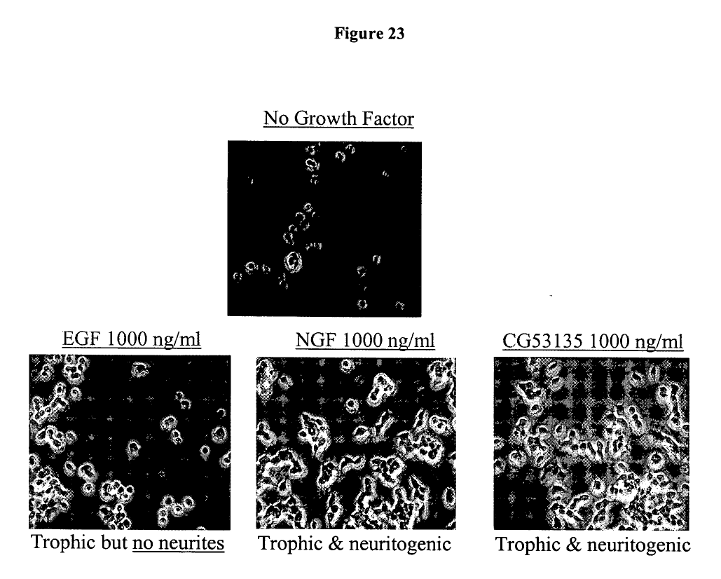

CG53135 induced neurite outgrowth in a dose-dependent fashion. The maximal extent of neurite outgrowth was achieved at 1000 ng/ml. The maximal extent of neurite outgrowth induced by both growth factors was equivalent. EGF did not induce neurite outgrowth. (FIG. 23)

### Conclusion:

CG53135 acts similarly to the neurotrophin NGF to the extent that CG53135 is capable of inducing similar neurite outgrowth. The capability of NGF to induce neurite outgrowth is an important feature of this growth factor that distinguishes it from other factors such as EGF which do not possess such neurotrophic activity. Thus, CG53135 possesses neurotrophic activity. Neurotrophic activity is recognized to have value in the treatment of numerous disorders of the central nervous system. In particular, the ability to induce neurite outgrowth is important to diseases where neurodegeneration is involved, such as Alzheimer's disease, Parkinson's disease and diseases with pathological structural changes or neural architecture are involved such as stroke and traumatic brain injury.

### 6.13 Example 13

### CG53135 Activates MAP Kinase in Neuronal PC12 Cells

This experiment was performed to assess the MAPK activating action of CG53135 and compare to the action of Nerve Growth Factor (NGF), Epidermal Growth Factor (EGF) and Basic FGF (bFGF).

### Materials and Method:

Materials: PC12 cells, tissue culture plates and medium (DMEM+/−10% FBS), NGF, EGF, bFGF, CG53135-05 E. coli purified product (by Process 1), EGF, MAPKK inhibitor PD98059

Method: Plate PC12 cells at medium density on poly-lysine coated tissue culture plates in DMEM+10% FBS. Culture 24 hours. Administer serum-free media containing NGF (100 ng/ml), EGF (100 ng/ml) or CG53135 (100 ng/ml) or no growth factor supplements. Pre-treat separate cultures with PD98059 before treating with CG53135 or NGF. Lyse cells 10 min post treatment, perform western blot with anti-phospho MAPK antibody to assess MAPK activation. Also, evaluate time course of MAPK activation by CG53135 and bFGF in human cortical neuronal cell line HCN1A at 0, 10 min, 1 hour and 3 hours.

### Results:

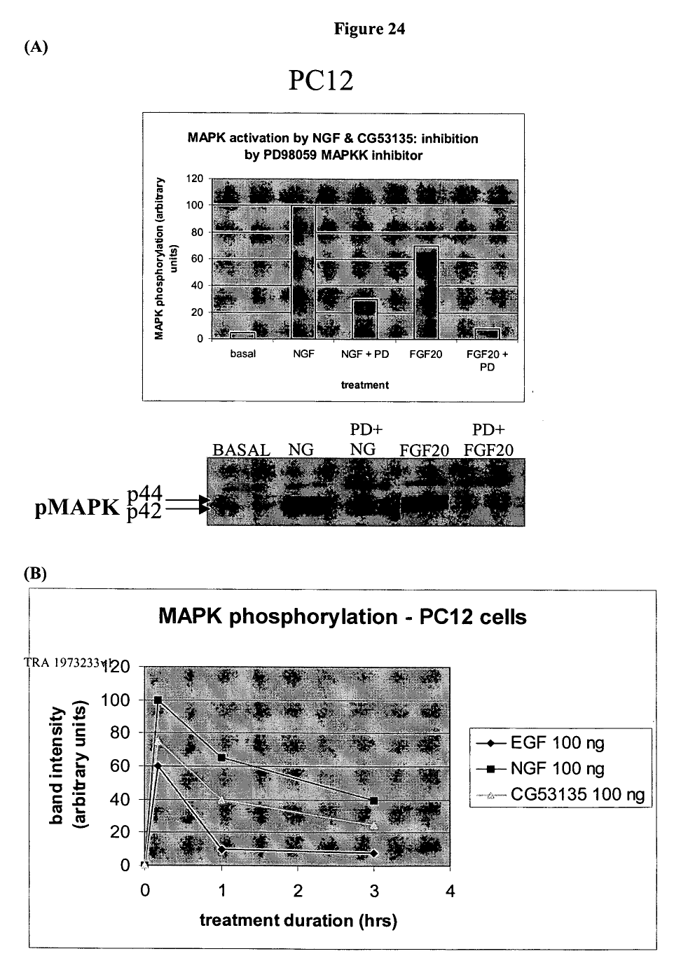

CG53135 induced robust MAPK activation in a MAPKK-dependent manner. CG53135 exhibits gradual and sustained MAPK activation timecourse, superior to bFGF. (FIG. 24)

### Conclusion:

CG53135 acts similarly to the neurotrophin NGF in the induction of MAPK, a key intracellular signaling molecule involved in cell survival and neuronal differentiation. The activation of this trophic pathway, which also is involved in processes underlying learning and memory, is recognized to have value in the treatment of numerous disorders of the central nervous system. In particular, the ability to protect neuronal cells is important to diseases where neurodegeneration is involved, such as Alzheimer's disease, Parkinson's disease and diseases with catastrophic cell death such as stroke and traumatic brain injury. The ability to stimulate intracellular pathways involved in learning and memory also is likely to have relevance to disorders involving memory dysfunction, such as Alzheimer's and age-related memory loss.

### 6.14 Example 14

### Manufacture of CG53135-05 and Pharmaceutical Formulations

Aiming for a construct that would be suitable for clinical development, untagged molecules were generated in a phage-free bacterial host. The codon-optimized, full-length, untagged molecule (CG53135-05) has the most favorable pharmacology profile and was used to prepare product for the safety studies and clinical trial.

6.14.1 Production Process and Pharmaceutical Formulations (Process 1)

CG53135-05 was expressed in Escherichia coli BLR (DE3) using a codon-optimized construct, purified to homogeneity, and characterized by standard protein chemistry techniques. The isolated CG53135-05 protein migrated as a single band (23 kilodalton) using standard SDS-PAGE techniques and stained with Coommassie blue. The CG53135-05 protein was electrophoretically transferred to a polyvinylidenefluoride membrane and the stained 23 kD band was excised from the membrane and analyzed by an automated Edman sequencer (Procise, Applied Biosystems, Foster City, Calif.); the N-terminal amino acid sequence of the first 10 amino acids was confirmed as identical to the predicted protein sequence.

### Fermentation and Primary Recovery Recombinant

CG53135-05 was expressed using Escherichia coli BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 30-40 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100˜110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

### Purification of the Drug Substance

The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 uM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

### Formulation and Fill/Finish

Four batches of purified drug substance were thawed for 24-48 hours at 2-8° C. and pooled into the collection tank of tangential flow ultrafiltration (TFF) equipment. The pooled drug substance was concentrated ˜5-fold via TFF, followed by about 5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at −80±15° C.

The finished drug product is a sterile, clear, colorless solution in single-use sterile vials for injection. CG53135-05 E. coli purified product was formulated at a final concentration of 8.2 mg/mL (Table 12).

TABLE 12
Composition of Drug Product

| Component | Grade | Final concentration | | Amount per Liter | |
|---|---|---|---|---|---|
| CG53135-05 E. coli | NA | 8.2 | mg/ml | 8.2 | g |
| purified product | | | | | |

Formulation Buffer

| | | | | | |
|---|---|---|---|---|---|
| Sodium acetate | USP | 40 | mM | 5.44 | g |
| (trihydrate) | | | | | |
| L-arginine HCl | USP | 200 | mM | 42.132 | g |
| Glycerol | USP | 3% | v/v | 30 | mL |

| Acetic acid | USP | NA | QS to pH 5.3 | |
|---|---|---|---|---|
| Water for injection | USP | NA | QS to 1 | L |

The pharmacokinetics of optimally-formulated CG53135-05 E. coli purified product was assessed in rats following intravenous, subcutaneous, and intraperitoneal administration to compare exposure at active doses in animal models and predict exposure in humans. Intravenous administration of CG53135-05 E. coli purified product resulted in high plasma levels (maximum plasma level=19,680-47,252 ng/mL), which rapidly declined within the first 2 hours to 30-70 ng/mL; decreased exposure was observed following the third daily dose (maximum plasma level=5373-7453 ng/mL). Subcutaneous administration of CG53135-05 E. coli purified product resulted in slow absorption (maximum plasma level at 10 hours) and plasma levels of 40-80 ng/mL up to 48 hours after dosing; some accumulation in plasma was seen following the third daily dose. Intraperitoneal administration of CG53135-05 E. coli purified product resulted in slow absorption (maximum plasma level at 2-4 hours) and plasma levels of 40-70 ng/mL up to 10 hours after dosing; decreased exposure was seen following third daily dose. No significant gender differences were observed by any route of administration.

Safety of intravenous administration of CG53135-05 E. coli purified product (0.05, 5 or 50 mg/kg/day (Bradford) for 14 consecutive days) was assessed in a pivotal toxicology study in rats. There were no treatment-related findings in rats administered 0.05 mg/mL (Bradford) CG53135-05 E. coli purified product for 14 days. In rats administered 5 mg/kg (Bradford) CG53135 for 14 days, food consumption was reduced and body weight was decreased; while there were no treatment-related changes in organ weights, urinalysis, opthalmology, or histopathology parameters in this dose group, there were treatment-related changes in hematology and clinical chemistry parameters in this treatment group. In rats administered 50 mg/kg (Bradford) CG53135-05 E. coli purified product for 12 days (estimated maximum plasma level of 20-30 fold higher than active dose), food consumption was reduced and body weight was markedly decreased; while there were no treatment-related changes in opthalmology, there were significant treatment-related changes in organ weights, urinalysis, hematology, clinical chemistry, and histopathology in this treatment group.

Safety of intravenous administration of CG53135-05 E. coli purified product (0 or 10 mg/kg/day (Bradford) for 7 consecutive days) was further assessed in a safety pharmacology study in rhesus monkeys. There were no treatment-related clinical observations in animals administered 1 mg/kg (Bradford) CG53135-05 E. coli purified product for 7 days. In animals administered 10 mg/kg (Bradford) CG53135-05 E. coli purified product for 7 days, minor effects on body weight were noted and associated with qualitative observations of lower food consumption. There were no apparent treatment-related effects on hematology, clinical chemistry, opthalmology, or electrophysiology in either dose group.

### Stability of CG53135-05 Drug Substances

Stability studies on the CG53135-05 E. coli purified product produced during cGMP manufacturing were performed. The analytical methods used as stability indicating assays for purified drug substance are listed in Table 13.

TABLE 13
Stability Assays for Drug Substance

| Assay | Stability Criteria |
|---|---|
| SDS-PAGE (Neuhoff stain) | >98% pure by densitometry |
| | (reduced and nonreduced) |
| RP-HPLC | Peak at 5.5 ± 1.0 min |
| | relative retention time |
| SEC-HPLC | >90% mono-disperse peak |
| Total protein by Bradford method | >0.2 mg/mL |
| Bioassay (BrdU) | PI₂₀₀ >0.5 ng/mL and <20 |
| | ng/mL |
| pH | 5.8 ± 0.4 |
| Visual appearance | Clear and colorless |

PI₂₀₀ = concentration of CG53135-05 that results in incorporation of BrdU at 2 times the background

The SDS-PAGE, RP-HPLC, and Bradford assays are indicative of protein degradation or gross aggregation. The SEC-HPLC assay detects aggregation of the protein or changes in oligomerization, and the bioassay detects loss of biological activity of the protein. The stability studies for the purified drug substance were conducted at −80 to 15° C. with samples tested at intervals of 3, 6, 9, 12, and 24 months.

In one experiment, stability studies of finished drug product were conducted by Cambrex at −80±15° C. and −20±5° C. with samples tested at intervals of 1, 3, 6, 9, 12, and 24 months. Stability data collected after 1 month indicate that finished drug product is stable for at least 1 month when stored at −80±15° C. or at −20±5° C. (Table 27).

TABLE 14
Stability Data for Drug Product after 1-month interval

| Assay | Stability Criteria | Initial | −80 [symbol] 15° C. | −20 [symbol] 5° C. |
|---|---|---|---|---|
| RP-HPLC | Major peak | Major peak | Major peak | Major peak |
| | retention time ± 0.2 | retention time ± | retention time ± | retention time ± |
| | min relative to | 0.2 min relative | 0.2 min relative | 0.2 min relative |
| | Reference | to Reference | to Reference | to Reference |
| | Standard | Standard | Standard | Standard |
| SDS-PAGE | Major band | Pass | Pass | Pass |
| | migrates at about | | | |
| | 23 kDa; | | | |
| | nonreduced minor | | | |
| | band below major | | | |
| | band | | | |
| SEC-HPLC | >90% mono-disperse | 100% | 100% | 100% |
| | peak | | | |
| Bradford | 10 [symbol] 0.2 mg/mL | 8.2 | 8.6 | 8.3 |
| Bioassay | PI₂₀₀ >0.5 ng/mL | 4.14 ng/mL | 2.98 ng/mL | 1/45 ng/mL |
| | and <20 ng/mL | | | |
| Sterility | Pass (i.e., no | Pass | NT | NT |
| | growth) | | | |
| pH | 5.3 ± 0.3 | 5.4 | 5.5 | 5.4 |
| Visual | Clear and colorless | Pass | Pass | Pass |
| appearance | solution | | | |

Lot #02502001 was stored at −80±15° C. or at −20±5° C. at Cambrex and tested after 1 month; PI200=concentration of CG53135-05 that results in incorporation of BrdU at 2 times the background; Pass=results met stability criterion; NT=not tested

In another experiment, samples of finished drug product were stored at −80±15° C. or stressed at 5±3° C., 25±2° C., or 37±2° C. and tested at various intervals for 1 month. Stability data indicate that finished drug product showed no significant instability after 1 month of storage at −80±15OC or 5±3° C. When stressed at 25±2° C., finished drug product was stable for at least 48 hours; degradation was apparent after 1 week at this temperature. When stressed at 37±2° C., degradation of finished drug product was apparent within 4 hours.

6.14.2 Improved Pharmaceutical Formulations and Production Process of CG53135-05 (Process 2)

A new formulation was developed to meet the three requirements for a commercial product: (1) the minimal storage temperature should be 2-8° C. for ease of distribution; (2) product should be stable at the storage temperature for at least 18 months for a commercial distribution system; and (3) product should be manufactured by commercial scale equipment, and processes should be transferable to various commercial contract manufacturers.

The new formulation consists 10 mg/mL (UV) of the protein product produced by the process described in Section 6.2 (“Process 2 protein”) in 0.5 M arginine as sulfate salt, 0.05 M sodium phosphate monobasic, and 0.01% (w/v) polysorbate 80. The lyophilized product is projected to be stable for at least 18 months at 2-8° C. based on accelerated stability data. In contrast to the new formulation, the previous formulation as described in U.S. application Ser. No. 10/435,087 is not possible to be lyophilized for the following reasons: firstly, the acidic component of the acetate buffer is acetic acid, which sublimes during lyophilization. After lyophilization, the loss of acetic acid is at 100% level with the basic component, sodium acetate, being the only buffering agent. This loss of acetic acid to lyophilization increases the pH to >7.5, which is far from the target pH of 5.3. Secondly, the glycerol has a collapse temperature of <−45° C., which renders this formulation not be able to be lyophilized commercially. Most of the commercial lyophilizers have a shelf temperature ranged from −45° C. to −50° C. with temperature variation of ±3° C.

Four unexpected properties of CG53135 were discovered and used to develop the new formulation: (1) high concentration of arginine, >0.4 M, increases the solubility to >30 mg/mL; (2) the use of sulfate salt of arginine increases the solubility by at least 2-6 fold; (3) the optimal concentration of sodium phosphate as a buffering salt is 50 mM, with a solubility of at least 1-2 fold increase in comparison with concentrations at 25, 75, and 100 mM; and (4) adding a surfactant during the diafiltration/ultrafiltration step minimizes the formation of aggregates. In development of the lyophilized formulation, each component of the new formulation was evaluated for solubility individually. CG53135-05 was precipitated using the precipitate buffer (50 mM NaPi, 5 mM EDTA, 1 M L-Arginine HCl, 2.5 M (NH₄)₂SO₄. The precipitate was washed with 25 mM sodium phosphate buffer at pH 6.5 to remove the residual arginine and ammonium sulfate. The washed precipitate was then re-dissolved in the following respective buffers listed in the tables. The following are examples of data.

TABLE 15
High concentration of arginine, >0.4 M, increases the solubility
to >30 mg/mL

| Concentra- | | | | | |
|---|---|---|---|---|---|
| tion of | Solubility of Process 2 protein in mg/mL | | | | |
| Arginine (M) | Batch #1 | Batch #2 | Batch #3 | Batch #4 | Batch #5 |
| 0.05 | 0.7 | 0.6 | 0.5 | ND | ND |
| 0.10 | 1.4 | 0.6 | 1.2 | ND | ND |
| 0.15 | 2.2 | 1.6 | 2.2 | ND | ND |
| 0.20 | 3.0 | 4.7 | 4.3 | ND | ND |
| 0.30 | ND | ND | ND | 5.8 | ND |
| 0.35 | ND | ND | ND | 10.1 | ND |
| 0.40 | ND | ND | ND | 9.8 | ND |
| 0.45 | ND | ND | ND | 32.3 | ND |
| 0.50 | ND | ND | ND | 23.8* | 37 |

*The solubility was lower as there was not sufficient protein in the experiment to be dissolved

TABLE 16
The use of sulfate salt of arginine increases the solubility by at least
2-6 folds.

| Concentra- | | | | | |
|---|---|---|---|---|---|
| tion | | | | | |
| of sodium | | | | | |
| phosphate | Solubility of Process 2 protein in mg/mL | | | | |
| monobasic* | Batch #A | Batch #B | Batch #C | Batch #D | Batch #E |
| 100 mM | 3.78 | 2.8 | 2.4 | 2.9 | 2.47 |
| 75 mM | 4.06 | 2.5 | 2.6 | 3.0 | 2.38 |
| 50 mM | 5.47 | 4.7 | 3.3 | 4.3 | 4.81 |
| 25 mM | 4.01 | 2.4 | 2.6 | 2.4 | 3.59 |

All formulation contains 0.2 M arginine.

An optimal concentration of the sodium phosphate as a buffering salt was observed (Table 17). The optimal concentration of sodium phosphate is 50 mM with a solubility of at least 1-2 fold increase in comparison with concentrations at 25, 75, and 100 Mm.

TABLE 17
The optimal concentration of sodium phosphate
as a buffering salt is 50 mM

| | Solubility Increament of | |
|---|---|---|
| | Process 2 protein in using | |
| | Arginine Sulfate vs Arginine | |
| | Phosphate in mg/mL | |
| Formulation | Batch #K | Batch # J |
| 50 mM sodium phosphate monobasic and | 4.4 | 2.3 |
| 0.15M Arginine at pH 7 | | |
| 50 mM sodium phosphate monobasic and | 6.5 | 5.2 |
| 0.15M Arginine at pH 7 | | |

Table 18 shows a need to add a surfactant during the diafiltration/ultrafiltration step to minimize the formation of aggregates. The experiment was conducted by performing the ultrafiltration/diafiltration at 2.5 mg/mL CG53135-05 in 0.2M arginine and 0.05 M sodium phosphate buffer at pH 7.0. After exchanging with 7 volumes of the final buffer (0.5M arginine and 0.05 M sodium phosphate buffer at pH 7.0), the diafiltrate is concentrated to ˜20 mg/mL. The diafiltrate is then diluted with the final buffer to ˜12.5 mg/mL and lyophilized. Polysorbate 80 is added either before or after the diafiltration to a final concentration of 0.01%.

TABLE 18
Adding a surfactant during the diafiltration/ultrafiltration
step minimizes the formation of aggregates.

| Polysorbate added | | |
|---|---|---|
| during | Process 2 | |
| ultrafiltration/ | protein Concentration | Turbidity |
| diafiltration | (mg/mL) | (NTU) |
| Yes | 12.5 | 20.9 |
| No | 13.0 | 4.6 |

All formulation contains 0.5 M arginine, 0.05 M sodium phosphate monobasic, and 0.01% polysorbate 80.

The new formulation has the following advantages: (1) a lyophilized product with a storage temperature of 2-8° C.; (2) a lyophilized product with a projected shelf-life of at least 18 months when stored at 2-8° C. achieve the solubility of >30 mg/mL; and (3) The lyophilized product has a collapse temperature of −30° C. which can be easily lyophilized by the commercial equipment. The interactions between arginine, sulfate, phosphate, and surfactant and CG53135 were unexpected.

The improved process steps for the manufacturing of drug substance and drug product are described in Table 19, and each step is explained below.

Manufacturing Process
Ampoule from WCB
↓
Seed Flask and Seed Fermenter 25 L - Innoc
↓
Fermentation at 1500 L scale
↓
Homogenization + 0.033% PEI or a charged
heterogenous polymer
↓
Purification by SP Streamline
↓
Purification by PPG 650M
↓
Cuno Filtration
↓
Purification by Phenyl Sepharose HP
↓
Concentration/Diafiltration addition of 0.01%
polysorbate 80 or Polysorbate 20
↓
Bottling - Drug Substance
↓
QC Testing and Release
↓
Sterile Vial Fill & Lyophilization
↓
Drug Product
↓
QC Testing and Release

Cell Bank: a Manufacturing Master Cell Bank (MMCB) in animal component free complex medium was used in an earlier Process. A second Manufacturing Master Cell Bank (MMCB) in animal component free chemically-defined medium was derived from the first MMCB and a Manufacturing Working Cell Bank (MWCB) was made from the second MMCB. This MWCB was used in the manufacturing process as described in Table 19.

Inoculum Preparation: the initial cell expansion occurs in shake flasks. Seed preparation is done by thawing and pooling 2-3 vials of the MWCB in chemically defined medium and inoculating 3-4 shake flasks each containing 500 mL of chemically-defined seed medium.

Seed and Final Fermentation: the shake flasks with cells in exponential growth phase (2.5-4.5 OD600 units) are used to inoculate a single 25 L (i.e., working volume) seed fermenter containing the seed medium. The cells upon reaching exponential growth phase (3.0-5.0 OD600 units) in the 25 L seed fermenter are transferred to a 1500 L production fermenter with 780-820 L of chemically defined batch medium. During fermentation, the temperature is controlled at 37±2° C., pH at 7.1±0.1, agitation at 150-250 rpm and sparging with 0.5-1.5 (vvm) of air or oxygen-enriched air to control dissolved oxygen at 25% or above. Antifoam agent (Fermax adjuvant 27) is used as needed to control foaming in the fermenter. When the OD (at 600 nm) of culture reaches 25-35 units, additional chemically defined medium is fed at 0.7 g/kg broth/min initially and then with feed rate adjustment as needed. The induction for expression of CG53135-05 protein is started when OD at 600 nm reaches 135˜165 units. After 4 hours post-induction the fermentation is completed. The final fermentation broth volume is approximately 1500 L. The culture is then chilled to 10-15° C.

Homogenization: the chilled culture is diluted with cell lysis buffer at the ratio of one part of fermentation broth to two parts of cell lysis buffer (50 mM sodium phosphate, 60 mM EDTA, 7.5 mM DTT, 4.5 M urea, pH 7.2. Polyethyleneimine (PEI), a flocculating agent is added to the diluted fermentation broth to a final PEI concentration at 0.033% (W/V). The cells are lysed at 10-15° C. with 3 passages through a high-pressure homogenizer at 750-850 bar.

Capture and Recovery: the chilled cell lysate is directly loaded in the upflow direction onto a pre-equilibrated Streamline SP expanded bed cation exchange column. During the loading, the bed expansion factor is maintained between 2.5-3.0 times the packed bed column volume. After loading, the column is flushed with additional Streamline SP equilibration buffer (100 mM sodium phosphate, 40 mM EDTA, 10 mM sodium sulfate, 3 M urea, pH 7.0) in the upflow direction. The column is then washed further with SP Streamline wash buffer (100 mM sodium phosphate, 5 mM EDTA, 25 mM sodium sulfate, 2.22 M dextrose, pH 7.0) in the downflow direction. The protein is eluted from the column with Streamline SP elution buffer (100 mM sodium phosphate, 5 mM EDTA, 200 mM sodium sulfate, 1 M L-arginine, pH 7.0) in the downflow direction.

PPG 650M Chromatography: the SP Streamline eluate is loaded on to a pre-equilibrated PPG 650 M, hydrophobic interaction chromatography column. The column is equilibrated and washed with 100 mM sodium phosphate, 200 mM sodium sulfate, 5 mM EDTA, 1 M Arginine pH 7.0. The column is further washed with 100 mM sodium phosphate, 5 mM EDTA, 0.9 M Arginine, pH 7.0. The product is eluted with 100 mM sodium phosphate, 5 mM EDTA, 0.2 M Arginine, pH 7.0.

CUNO Filtration: the PPG eluate is passed through an endotoxin binding CUNO 30ZA depth filter. The filter is flushed first with water for injection (WFI) and then with 100 mM sodium phosphate, 5 mM EDTA, 0.2 M Arginine, pH 7.0 (PPG eluate buffer). After flushing, the PPG eluate is passed through the filter. Air pressure is used to push the final liquid through the filter and its housing.

Phenyl Sepharose Chromatography: the CUNO filtrate is then loaded on to a pre-equilibrated Phenyl Sepharose hydrophobic interaction chromatography column. The column is equilibrated and washed with 100 mM sodium phosphate, 50 mM ammonium sulfate, 800 mM sodium chloride, 0.5 M Arginine, pH 7.0. The product is eluted with 50 mM sodium phosphate, 0.5 M Arginine, pH 7.0.

Concentration and Diafiltration: a 1% Polysorbate 80 is added to the Phenyl Sepharose eluate so that the final concentration in the drug substance is 0.01% (w/v). The eluate is then concentrated in an ultrafiltration system to about 2-3 g/L. The retentate is then diafiltered with 7 diafiltration volumes of 50 mM sodium phosphate, 0.5 M Arginine, pH 7.0 (Phenyl Sepharose elution buffer). After diafiltration the retentate is concentrated between 12-15 g/L. The retentate is filtered through a 0.22 μm filter and subsequently diluted to 10 g/L.

Bulk Bottling: the retentate from the concentration and diafiltration step is filtered through a 0.22 μm pore size filter into 2 L single use Teflon bottles. The bottles are frozen at −70° C.

Drug Product/Vial: the Frozen Drug Substance is transported to Formatech Inc, MA from Diosynth—RTP, NC for the manufacture of the Drug Product. The bottles of frozen Drug Substance are thawed at ambient temperature. After the Drug Substance is completely thawed, it is pooled in a sterile container, filtered, filled into vials, partially stoppered, and lyophilized. After completion of the freeze-drying process, the vials are stoppered and capped. The lyophilized Drug Product is stored at 2-8° C.

The CG53135-05 reference standard was prepared at Diosynth RTP Inc, using a 140L scale manufacturing process that was representative of the bulk drug substance manufacturing process (as described in the General Method of Manufacture). The reference standard was stored as 1 mL aliquots in 2 mL cryovials at −80° C.±15° C.

Purity of the final product was analyzed by SDS-PAGE, RP-HPLC, size exclusion-HPLC, and Western blot. Potency of the drug was measured by growth of NIH 3T3 cells in response to CG53135-05. All data indicated that the final product is suitable for clinical uses.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

What is claimed is:

1. A method of preventing or treating arthritis or cartilage degeneration comprising administering to a subject in need thereof an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

2. A method of preventing or treating arthritis or cartilage degeneration comprising administering to a subject in need thereof an effective amount of a composition comprising a protein isolated from a cultured host cell containing an isolated nucleic acid molecule selected from the group consisting of:
(a) a nucleic acid molecule comprising a nucleotide sequence selected from the group consisting of SEQ ID NOs: 1, 3, 5, 6, 8, 9, 21, 23, 25, 27, 29, 31, 33, 35, 37, 39 and 41;
(b) a nucleic acid molecule encoding a protein comprising an amino acid sequence selected from the group consisting of SEQ ID NOs: 2, 4, 7, 10, 22, 24, 26, 28, 30, 32, 34, 36, 38, or 40; and
(c) a nucleic acid molecule hybridizes under stringent conditions a nucleotide sequence of SEQ ID NOs: 1, 3, 5, 6, 8, 9, 21, 23, 25, 27, 29, 31, 33, 35, 37, 39 and 41, or a complement of said nucleic acid molecule, and wherein said stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA.

3. The method of claim 2, wherein said host cell is a eukaryotic cell.

4. The method of claim 2, wherein said host cell is a prokaryotic cell.

5. The method of claim 4, wherein said prokaryotic cell is E. coli.

6. The method of claim 2, wherein said protein isolated from a cultured host cell has a purity of at least 98%.

7. A method of preventing or treating stroke or a neurodegenerative disease comprising administering to a subject in need thereof an effective amount of a composition comprising an isolated protein comprising an amino acid sequence of SEQ ID NOs:4, 7, 10, 22, 24, 26, 28, 30, 32, 34, 36, 38, or 40;

8. The method of claim 1, 2 or 7, wherein said composition further comprises a pharmaceutically acceptable carrier.

9. The method of claim 8, wherein said composition comprises 0.02-0.2 M acetate, 0.5-5% glycerol, 0.2-0.5 M arginine-HCl, and 0.5-5 mg/ml of said isolated protein.

10. The method of claim 9, wherein said composition comprises 0.04M sodium acetate, 3% Glycerol (volume/volume), 0.2M Arginine-HCl at pH 5.3, and 3 mg/ml of said isolated protein.

11. The method of claim 8, wherein said composition comprising 0.01-1 M arginine in a salt form, sulfobutyl ether Beta-cyclodextrin sodium, or sucrose, about 0.01-0.1 M sodium phosphate monobasic (NaH₂PO₄.H₂O), about 0.01%-0.1% weight/volume (“w/v”) polysorbate 80 or polysorbate 20, and about 0.005 mg/ml to about 50 mg/ml of said isolated protein.

12. The method of claim 11, wherein said composition comprises arginine in a salt form selected from the group consisting of arginine, arginine sulfate, arginine sulfone, and arginine hydrochloride.

13. The method of claim 11, wherein said wherein said arginine in a salt form, sulfobutyl ether Beta-cyclodextrin sodium or sucrose is of 0.01-0.7 M.

14. The method of claim 11, wherein said composition comprises an arginine in a salt form at a concentration of 0.5 M.

15. The method of claim 11, wherein said sodium phosphate monobasic is 0.05 M.

16. The method of claim 11, wherein said polysorbate 80 or polysorbate 20 is 0.01% (w/v).

17. The method of claim 11, wherein said isolated protein is at a concentration of 5-30 mg/ml.

18. The method of claim 11, wherein said isolated protein is at a concentration of 10 mg/ml.

19. The method of claim 11, wherein said isolated protein comprises two or more proteins.

20. The method of claim 19, wherein said composition comprises a first protein comprising an amino acid sequence of SEQ ID NO:24, and a second protein comprising an amino acid sequence of SEQ ID NO:2.

21. The method of claim 20, wherein said composition further comprises an isolated protein comprising an amino acid sequence selected from the group consisting of SEQ ID NOs:26, 28, 30 and 32.

22. The method of claim 20, wherein said composition further comprises a third protein comprising an amino acid sequence of SEQ ID NO:28, a fourth protein comprising an amino acid sequence of SEQ ID NO:30, and a fifth protein comprising an amino acid sequence of SEQ ID NO:32.

23. The method of claim 10, wherein said composition is lyophilized.

24. The method of claim 1, 2 or 7, wherein said subject is a mammal.

25. The method of any of claims 24, wherein said mammal is a human.

26. The method of claim 1, 2 or 7, wherein said administering is parenteral administration.

27. The method of claim 26, wherein said parenteral administration is intravenous administration.

28. The method of claim 26, wherein said parenteral administration is subcutaneous administration.

29. The method of claim 1, 2 or 7, wherein said administering is transdermal administration.

30. The method of claim 1, 2 or 7, wherein said administering is transmucosal administration.

31. The method of claim 30, wherein said transmucosal administration is nasal administration.

32. An isolated nucleic acid molecule selected from the group consisting of:
(a) a nucleic acid molecule comprising a nucleotide sequence selected from the group consisting of SEQ ID NOs: 23, 25, 27, and 29;
(b) a nucleic acid molecule encoding a protein comprising an amino acid sequence selected from the group consisting of SEQ ID NOs: 24, 26, 28, and 30;
(c) a nucleic acid molecule hybridizes under stringent conditions to a nucleotide sequence of SEQ ID NO: 23, 25, 27 or 29, or a complement of said nucleic acid molecule, and wherein said stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA;
(d) a fragment of an nucleic acid molecule of any of (a)-(c); and
(e) a complement of an nucleic acid molecule of any of (a)-(d).

33. The isolated nucleic acid molecule of claim 32 comprising SEQ ID NO:23.

34. A vector comprising the nucleic acid molecule of claim 32.

35. The vector of claim 34, wherein said nucleic acid molecule is operably linked to an expression control sequence.

36. A prokaryotic or eukaryotic host cell containing a nucleic acid molecule of claim 32.

37. A prokaryotic or eukaryotic host cell containing the vector of claim 34.

38. A prokaryotic or eukaryotic host cell containing the vector of claim 35.

39. A method comprising culturing the host cell of claim 37 or 38 in a suitable nutrient medium.

40. The method of claim 39, wherein said host cell is E. coli.

41. The method of claim 39 further comprising isolating a protein encoded by said nucleic acid molecule from said cultured cells or said nutrient medium.

42. An isolated protein by method of claim 41.

43. An isolated protein selected from the group consisting of:
(a) a protein comprising an amino acid sequence of SEQ ID NO: 24, 26, 28, or 30;
(b) a protein with one or more amino acid substitutions to the protein of (a), wherein said substitutions are no more than 15% of the amino acid sequence of SEQ ID NO: 24, 26, 28, or 30, and wherein said protein with one or more amino acid substitutions retains cell proliferation stimulatory activity;
(c) a fragment of the protein of (a) or (b); and
(d) a carbarmylated protein of (a)-(c).

44. The isolated protein of claim 43 comprising an amino acid sequence of SEQ ID NO:24.

45. An isolated protein comprising an amino acid sequence, wherein said amino acid sequence has one or more conservative amino acid substitutions relative to SEQ ID NO: 24, 26, 28, or 30.

46. A pharmaceutical composition comprising a pharmaceutically acceptable carrier, and a protein of any of claims 42-45.

47. A method of stimulating cartilage regeneration or repair comprising administering to a subject in need thereof an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.
